(19) United States
(12) Patent Application Publication
(10) Pub. No.: US 2004/0096967 A1
(43) Pub. Date: May 20, 2004

(54) Suspension method for producing embryoid bodies, compositions and methods related thereto

(75) Inventors: Timothy D. Gryseels, Ledyard, CT (US); John E. Hambor, Madison, CT (US); Steven J. Hawrylik, North Stonington, CT (US); Marsh L. Roach, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/698,840

(22) Filed: Oct. 31, 2003

Related U.S. Application Data
(60) Provisional application No. 60/422,834, filed on Oct. 31, 2002.

(51) Int. Cl. C12N 5/06; C12N 5/08

(52) U.S. Cl. 435/366; 435/354

(57) ABSTRACT

Suspension methods and compositions for effecting large-scale in vitro differentiation of mammalian embryonic stem cells are provided, as well as methods comprising the subject large-scale in vitro differentiation methods.

### CROSS REFERENCE TO RELATED APPLICATION

This application claims the benefit of U.S. Provisional Application No. 60/422,834 filed Oct. 31, 2002.

### FIELD OF THE INVENTION

The present invention relates to methods for producing embryoid bodies that are useful for production of in-vitro differentiated cell lines as well as for the production of tissues. These cell lines and tissues are useful in a variety of research situations, as well as for medical treatments. The present invention also relates to highly homogenous compositions of embryoid bodies and cells produced therefrom. The methods and compositions of the present invention are especially suitable for large-scale uses.

### INTRODUCTION

Stem cells, with their capability for self-regeneration in vitro and their ability to produce differentiated cell types, may be useful for replacing the function of aging or failing cells in nearly any organ system. By some estimates, over 100 million Americans suffer from disorders that might be alleviated by transplantation technologies that utilize stem cells (Perry, Science, 287:1423 (2000)). Such illnesses include, for example, cardiovascular diseases, autoimmune diseases, diabetes, osteoporosis, cancers and burns. Many nervous system disorders, such as Parkinson's, Alzheimer's, multiple sclerosis, and other neurodegenerative diseases, may only be able to be treated by replacement of the lost or damaged nervous tissue cells. Tissue replacement therapy may also prove useful for repairing cardiac tissue destroyed by heart infarction, for replacing damaged cartilage in such disorders as osteoarthritis, for replacing defective pancreatic islet cells in those afflicted with diabetes, and for replacing lost blood and immune cells lost due to cancer and immune diseases. Tissue transplantation or replacement therapy is generally limited by the availability of tissues as well as the risk of transplant rejection. Such problems could be obviated by tissue production techniques involving the use of totipotent or pluripotent stem cells, such as embryonic stem (ES) cells.

ES cells were first isolated in the 1980's by several independent groups. They are able to differentiate to virtually all cell and tissue types in vivo as well as in vitro. In 1986, Gossler (Gossler et al., PNAS, 83: 9065-69 (1986)) described the ability and advantages of using ES cells to produce transgenic animals. The next year, Thomas and Capecchi (Cell, 51:503 (1987)) reported the ability to alter the genome of the ES cells by homologous recombination. Smithies et al. later demonstrated that ES cells modified by gene targeting when re-introduced into blastocysts could transmit the genetic modifications through the germline. Today, genetic modification of the murine genome by ES cell technology is a seminal approach to understanding the function of mammalian genes in vivo. ES cells have been isolated from other mammalian species (for example, hamster, rat, mink, pig, cow), but to date only murine ES cells have successfully transmitted the ES cell genome through the germline. Recently, interest in stem cell technology has intensified with the reported isolation of primate and human ES cells.

ES cells are typically isolated from the inner cell mass of the blastocyte stage embryo, and if maintained in optimal conditions, will continue to grow indefinitely in an undifferentiated, diploid state. ES cells are sensitive to pH changes, overcrowding, and temperature changes, making culture of these cells difficult. ES cells that are not cultured properly will spontaneously differentiate. The full differentiation potential of ES cells may be reached only if the ES cells are forced to differentiate both by morphological cues, such as embryoid body (EB) formation, and by the infusion of growth- and differentiation-inducing factors. Steps for the induction of ES cell differentiation include the production of morula-like aggregates, followed by the formation of EB in suspension cultures, and final differentiation in adhesion culture.

Current protocols for the generation of EB are either of limited productivity (can only be done small scale) or deliver EB with a large variation in size and differentiation state. In order to generate large cultures of ES cells suitable for differentiation into transplantation-quality tissues, it is important to first establish controllable methods for producing large amounts of suitable EB.

### SUMMARY OF THE INVENTION

The present invention provides suspension methods for producing embryoid bodies, as well as cellular compositions of such embryoid bodies. In one embodiment, the method for the formation of embryoid bodies comprises inoculating a culture vessel with a culture of undifferentiated embryonic stem cells, wherein said culture vessel contains a medium suitable for inducing embryoid body formation, and incubating said culture vessel while subjecting it to shaking. In certain embodiments, said shaking is accomplished through rotary shaking. Control of the characteristics of the EB may be accomplished through the rate of shaking of an ES cell suspension culture in a culture vessel during the EB formation stage.

In one aspect, the invention provides methods for effecting large-scale in vitro differentiation of ES cells which incorporate the suspension methods for producing embryoid bodies (EB). In another aspect, the invention provides methods for effecting large-scale in vitro differentiation of ES cells from cellular compositions comprising EB. Large-scale cultures of differentiated ES cells produced by the methods of the invention are expected to have improved phenotype, size, and viability, and may thus be more suitable for use in tissue transplantation and cell replacement therapies. In one embodiment, the method for differentiating ES cells comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating the resulting embryoid bodies, inoculating the dissociated embryoid bodies into a medium suitable for accomplishing further differentiation, and culturing said dissociated embryoid bodies to a later differentiation state.

The above methods for effecting large-scale in vitro differentiation of ES cells may, for example, be incorporated in methods of screening therapeutic agents, producing cells for cell replacement therapy or producing tissues for use in tissue transplantation, as well as gene expression profiling. Such methods are also encompassed within the present invention.

The invention also provides cellular compositions comprising embryoid bodies. Such cultures may be suitable for use in methods of large-scale in vitro differentiation of ES cells or in other methods comprising in vitro differentiation of ES cells such as the production of cells, tissues, or gene products. The ES cells produced by such methods may be used, for example, in gene therapy comprising the use of stem cells, or to produce transgenic animals.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds., 1984); Transcription And Translation (B. D. Hames & S. J. Higgins eds., 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., Cold Spring Harbor Laboratory, 1987); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “agonist” is art-recognized and refers to a compound that mimics or up-regulates (e.g., potentiates or supplements) the bioactivity of a protein. An agonist may be a wild-type protein or derivative thereof having at least one bioactivity of the wild-type protein. An agonist may also be a compound that upregulates expression of a gene or which increases at least one bioactivity of a protein. An agonist may also be a compound which increases the interaction of a polypeptide with another molecule, e.g., a target peptide or nucleic acid.

The term “amino acid” is intended to embrace all molecules, whether natural or synthetic, which include both an amino functionality and an acid functionality and that are capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing. The names of the natural amino acids are abbreviated herein in accordance with the recommendations of IUPAC-IUB.

The term “antagonist” is art-recognized and refers to a compound that downregulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that downregulates expression of a gene or which reduces the amount of expressed protein present.

The terms “antibiotic”, “germicide” and “antimicrobial” are art-recognized and refer to agents or molecules capable of killing, inactivating, or otherwise neutralizing the pathogenic or reproductive ability of microorganisms. Terms such as “bactericides”, “viricides” or “antivirals”, “antifungals”, “antihelmintics” and the like refer to categories of such agents which have the ability to kill, inactivate, or otherwise neutralize the pathogenic or reproductive ability of bacteria, viruses, fungi, and various parasites, respectively.

“Biomarker” refers to a biological molecule whose presence, concentration, activity, or phosphorylation state may be detected and used to identify the phenotype of a cell.

The terms “cell culture” or “culture” include any combination of cells and medium. The cells need not be actively growing.

The term “cellular composition” refers to a preparation of cells, which preparation may include, in addition to the cells, non-cellular components such as cell culture media, e.g., proteins, amino acids, nucleic acids, nucleotides, co-enzyme, anti-oxidants, metals and the like. Furthermore, the cellular composition can have components which do not affect the growth or viability of the cellular component, but which are used to provide the cells in a particular format, e.g., as polymeric matrix-for encapsulation or a pharmaceutical preparation.

The term “culture” refers to any growth of cells, organisms, multicellular entity, or tissue in a medium. The term “culturing” refers to any method of achieving such growth, and may comprise multiple steps. The term “further culturing” refers to culturing a cell, organism, multicellular entity, or tissue to a certain stage of growth, then using another culturing method to bring said cell, organism, multicellular entity, or tissue to another stage of growth. For example, in certain of the methods provided herein, undifferentiated embryonic stem cells are cultured to the embryoid body stage. The embryoid bodies are then further cultured into differentiated stem cells. A “cell culture” refers to a growth of cells in vitro. In such a culture, the cells proliferate, but they do not differentiate or organize into tissue per se. A “tissue culture” refers to the maintenance or growth of tissue, e.g., explants of organ primordial or of an adult organ in vitro so as to preserve its architecture and function.

Tissue and cell culture preparations of the subject micro-organ explants and amplified progenitor cell populations can take on a variety of formats. Some non-limiting examples of such are as follows. A “monolayer culture” refers to a culture in which cells multiply in a suitable medium while mainly attached to each other and to a substrate. A “suspension culture” refers to a culture in which cells multiply while suspended in a suitable medium. A “continuous flow culture” refers to the cultivation of cells or explants in a continuous flow of fresh medium to maintain cell growth, e.g., viability. The term “conditioned media” refers to the supernatant, e.g., free of the cultured cells/tissue, resulting after a period of time in contact with the cultured cells such that the media has been altered to include certain paracrine and/or autocrine factors produced by the cells and secreted into the culture. A “confluent culture” is a cell culture in which all the cells are in contact and thus the entire surface of the culture vessel is covered, and implies that the cells have also reached their maximum density, though confluence does not necessarily mean that division will cease or that the population will not increase in size.

In general, a culture method may comprise the following steps. The cells, organisms, multicellular entity, or tissue are inoculated and grown in a medium until all of the available substrate is occupied or the capacity of the medium is reached. At this stage, the medium is changed or the culture is divided, or “subcultured”. For adherent cells, subculturing usually involves removal of the medium and dissociation of the cells in the monolayer with trypsin, although loosely adherent cells may be subcultured by shaking the bottle, collecting the cells in the medium, and diluting them into fresh medium.

The term “culture medium” or “medium” is recognized in the art, and refers generally to any substance or preparation used for the cultivation of living cells. The term “medium suitable for inducing embryoid body formation” refers to a substance or preparation suitable for the cultivation of embryoid bodies from undifferentiated embryonic stem cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. In other words, a nutrient rich liquid prepared for bacterial culture is a medium. Similarly, a powder mixture that, when mixed with water or other liquid, becomes suitable for cell culture, may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD₆₀₀ of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, Mc Coy's 5A, Dulbecco's MEM/F-I 2, RPMI 1640, and Iscove's Modified Dulbecco's Medium (IMDM).

The terms “comprise” and “comprising” are used in the inclusive, open sense, meaning that additional elements may be included.

The term “culture vessel” includes any vessel suitable for holding a liquid cell culture. Many culture vessels are known in the art. Exemplary culture vessels include fermentors, Erlenmeyer flasks, baffled flasks, Tunac-type flasks (e.g., the Tunair flask), 96-well plates (or other multi-chambered systems), beakers, bags, test tubes, Fernbach flasks, etc. A “simple culture vessel” is a culture vessel that is not equipped for providing a partial pressure of oxygen that exceeds that of the room (or other general surroundings) in which the simple culture vessel is located. Examples of simple culture vessels include Erlenmeyer flasks, baffled flasks, Tunac-type flasks, 96-well plates, beakers, bags, test tubes, Fernbach flasks, etc. Many simple culture vessels do not have any means for mixing the culture. Such vessels may be termed “externally agitated culture vessels”, and cultures contained in externally agitated culture vessels are generally mixed by attaching the flask to a device that agitates the culture, for example by providing orbital motion, back and forth motion or rocking motion. Most often, external agitation is provided by attaching the flask to an orbital shaker table.

The term “differentiation” refers to the formation of cells expressing markers or exhibiting functional attributes known to be associated with cells that are more specialized and closer to becoming terminally differentiated cells incapable of further division or differentiation. For example, in a pancreatic context, differentiation can be seen in the production of islet-like cell clusters containing an increased proportion of beta-epithelial cells that produce increased amounts of insulin. The terms “further” or “greater” differentiation refers to cells that are more specialized and closer to becoming terminally differentiated cells incapable of further division or differentiation than the cells from which they were cultured. The term “final differentiation” refers to cells that have become terminally differentiated cells incapable of further division or differentiation.

A “diseased” cell as used herein refers to a cell taken from an individual that has a disease or disorder.

The term “embryoid body” or “EB” refers to any multicellular entity that results when pluripotent cells derived from a blastocyst are further differentiated.

“Gene” or “recombinant gene” refer to a nucleic acid molecule comprising an open reading frame and including at least one exon and (optionally) an intron sequence. “Intron” refers to a DNA sequence present in a given gene which is spliced out during mRNA maturation.

“Gene construct” refers to a vector, plasmid, viral genome or the like which includes a “coding sequence” for a polypeptide or which is otherwise transcribable to a biologically active RNA (e.g., antisense, decoy, ribozyme, etc), may transfect cells, in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “gene product” refers to a molecule that is produced as a result of transcription of a gene. Gene products include RNA molecules transcribed from a gene, as well as proteins translated from such transcripts.

The term “growth factor” refers to any one of a number of biochemical substances that stimulates cell division upon contacting cells. Growth factors include, for example hormones, proteins, peptides, cytokines, and the like.

The term “high throughput screening” refers to an iterative process of screening a sample of compounds for activity, analyzing the results, and selecting a new set of compounds for screening, based on what has been learned from the previous screens. The term may be also used to refer to any process in which batches of compounds are tested for binding activity or biological activity against target molecules, cells, organisms, or tissues. Test compounds may act as inhibitors of target enzymes, as competitors for binding of a natural ligand to its receptor, as agonists or antagonists for receptor-mediated intracellular processes, and so forth. In general, high-throughput screening is used to screen large numbers of compounds rapidly and in parallel.

“Host cell” refers to a cell transduced with a specified transfer vector. The cell is optionally selected from in vitro cells such as those derived from cell culture, ex vivo cells, such as those derived from an organism, and in vivo cells, such as those in an organism. “Recombinant host cells” refers to cells which have been transformed or transfected with vectors constructed using recombinant DNA techniques. “Host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “including” is used herein to mean “including but not limited to”. “Including” and “including but not limited to” are used interchangeably.

The term “incubating” refers to the process of maintaining constant temperature, humidity, and other environmental factors for the purpose of allowing a microbe, cell, multicellular entity, or tissue culture to grow. Constant temperature, humidity, and other environmental factors may be maintained using an “incubator”, or device able to control these factors.

The terms “library” or “combinatorial library” refer to a plurality of molecules, which may be termed “members,” synthesized or otherwise prepared from one or more starting materials by employing either the same or different reactants or reaction conditions at each reaction in the library. In general, the members of any library show at least some structural diversity, which often results in chemical and biological diversity. Such structural diversity in preparing libraries of coordination molecules may include, by way of example, metal ion diversity, ligand diversity, solvation diversity or counter-ion diversity. A library may contain any number of members from two different members to about 10⁸ members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase. Further details regarding the libraries of the present invention are described below.

The term “lineage committed cell” refers to a progenitor cell that has been induced to differentiate into a specific cell type, e.g., a pancreatic cell.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides. Exemplary nucleic acids for use in the subject invention include antisense, decoy molecules, recombinant genes (including transgenes) and the like. The term “nucleic acid” encompasses “aptamers”, which are single-stranded nucleic acid molecules that have been developed to bind a molecular target, usually by in vitro selection methods.

The term “organ” refers to two or more adjacent layers of tissue, which layers of tissue maintain some form of cell-cell and/or cell-matrix interaction to form a microarchitecture.

A “patient”, “subject” or “host” to be treated by the subject method may mean either a human or non-human animal.

The term “phenotype” refers to the observable characteristics of a cell, such as size, morphology, protein expression, etc.

“Protein”, “polypeptide” and “peptide” are used interchangeably herein when referring to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

The term “primary culture” denotes a mixed cell population of cells that permits interaction of many different cell types isolated from a tissue. The word “primary” takes its usual meaning in the art of tissue culture. For example, a primary culture of pancreatic duct cells may allow the interaction between mesenchymal and epithelial cells.

The term “progenitor cell” refers to a cell that has the capacity to create progeny that are more differentiated than itself. For example, the term may refer to an undifferentiated cell or cell differentiated to an extent short of final differentiation which is capable of proliferation and giving rise to more progenitor cells having the ability to generate a large number of mother cells that can in turn give rise to differentiated, or differentiable daughter cells. In a preferred embodiment, the term progenitor cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. By this definition, stem cells may also be progenitor cells, as well as the more immediate precursors to terminally differentiated cells.

“Proliferation” refers to an increase in cell number. “Proliferating” and “proliferation” refer to cells undergoing mitosis.

The term “rotary shaker” refers to a device capable of creating fluid rotational motion in tubes and vessels. Examples of rotary shakers include, but are not limited to, the INFORS Orbitec, Wavetec, Labotron and other INFORS shakers, VWR Signature Digital Waver, Orbital Shaker, Platform Gyratory Shaker, Rocker, and other VWR shakers, G25 New Brunswick rotary shakers, and the like.

“Small molecule” refers to a compound having a molecular weight of no more than about 20 kDa. Small molecules may be nucleic acids, peptides, peptide-nucleic acids, aptamers polypeptides, peptidomimetics, or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify molecules that bind a microorganism.

“Stem cell” or “pluripotent stem cell” is art-recognized, and refers to a cell, capable of both indefinite proliferation and differentiation into specialized cells, that serves as a continuous source of new cells. The term “embryonic stem cell” or “ES cell”, refers to cultured cells obtained by isolation of inner cell mass cells from blastocysts or by isolation of primordial germ cells from a fetus. The term “undifferentiated embryonic stem cells” refers to embryonic stem cells not yet intentionally induced to differentiate.

The term “substantially pure”, with respect to progenitor cells, refers to a population of progenitor cells that is at least about 75%, preferably at least about 85%, more preferably at least about 90%, and most preferably at least about 95% pure, with respect to progenitor cells making up a total cell population. Recast, the term “substantially pure” refers to a population of progenitor cell of the present invention that contain fewer than about 20%, more preferably fewer than about 10%, most preferably fewer than about 5%, of lineage committed cells in the original unamplified and isolated population prior to subsequent culturing and amplification.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating red cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

The term “tissue” refers to a group or layer of similarly specialized cells which together perform certain special functions.

2. Suspension Culture of Embryoid Bodies

In part, the invention provides suspension methods for producing embryoid bodies. In one embodiment, the method for the formation of embryoid bodies comprises inoculating a culture vessel with a culture of undifferentiated embryonic stem cells, wherein said culture vessel contains a medium suitable for inducing embryoid body formation, and incubating said culture vessel while subjecting it to shaking. In certain embodiments, said shaking is accomplished through rotary shaking.

The generation of large cultures of differentiated cells suitable for cell therapy or tissue production is in part dependent on developing controllable suspension methods for producing cultures of EB suitable for use in such large-scale in vitro differentiation methods. Such cultures are expected to have superior characteristics to those produced by other methods in the art, having, for example, a more uniform phenotype, a larger diameter, improved viability, and the like. Control of the characteristics of the EB may be accomplished through controlling the rate of shaking of an ES cell suspension culture in a shaker flask during the EB formation stage, as well as the number of cells used to inoculate the culture, the time of incubation, and size and volume of the culture vessel.

Shaking of the subject suspension cultures may be achieved by using a simple culture vessel that does not have any integrated means for agitating the culture. Such vessels are termed “externally agitated culture vessels”. Non-limiting examples of such vessels include Erlenmeyer flasks, baffled flasks, Tunac-type flasks, 96-well plates, beakers, bags, test tubes, conical flasks, Fernbach flasks, etc. Bioreactors may also be used as culture vessels provided the culture within may be shaken. In one embodiment, the shaker flask is a Fernbach flask. A variety of systems for agitating an externally agitated culture vessel are available. For example, a vessel may be attached to a device that agitates the culture, such as a device that provides one or more of orbital motion (horizontal or vertical), back and forth motion or rocking motion. In certain embodiments, external agitation is provided by attaching the vessel to an orbital shaker table. Optionally, a method of the invention may employ an orbital shaker table with an orbital radius of one inch and a rotational speed of 200 to 300 revolutions per minute.

Generally, agitation speed and orbit affect both aeration and the mixing of the culture. Greater aeration increases oxygen transfer rate. The size of the orbit affects gas transfer rates linearly and the speed has a square-law relationship. Therefore, if the size of the orbit doubles, say from ½″ to 1″, the gas transfer rate should double. If the speed doubles, the gas transfer should quadruple. Of course, these are general rules and certain flask shapes or other conditions may cause deviation from predicted effects.

In certain embodiments, it is desirable to manipulate the rate at which one or more gases dissolve into a culture (the gas transfer rate). Many cells grow more rapidly or to a greater density if the culture is supplied with an increased oxygen transfer rate. Alternatively, certain cells, particularly anaerobic cells, grow more rapidly or to a greater density if the culture is depleted of oxygen. The transfer rate of a gas into a liquid culture is determined by a host of variables, including the surface area between the gas and the liquid, the relative movement of liquid and gas past each other, the temperature, the partial pressure of the gas already dissolved in the liquid, the partial pressure of the gas in the gaseous phase, etc. These variables may be manipulated in a variety of ways. For example, fermentors are typically equipped with a pressurized supply of oxygen, and the culture is maintained at a high pressure so that a very high oxygen transfer rate and partial pressure of dissolved oxygen is obtained. In methods that employ a simple culture vessel, oxygen transfer rate may be manipulated by, for example, selecting a vessel shape and culture volume that provide a desired surface area, by agitating the culture, or by placing the simple culture vessel in a room or other enclosed space that is supplied with a partial pressure of oxygen that is greater or less than the average atmospheric partial pressure of oxygen.

Oxygen transfer rate (also sometimes termed “aeration rate”) may be measured by any of a variety of methods known in the art. One general approach to assessing the oxygen transfer rate involves operating the cell culture system under conditions similar to those used for actual cultures, but the culture vessel contains a liquid having a reagent that readily reacts with oxygen to create a measurable effect. By tracking the amount of reagent that reacts with oxygen over time in the culture system, an oxygen transfer rate can be determined. The oxygen transfer rate reflects the rate at which oxygen may be supplied to cells in culture. Oxygen absorption rates are commonly expressed in terms of millimoles of oxygen/liter/minute. For example, oxygen transfer rates may be measured by the sulfite oxidation method described in U.S. Pat. No. 4,665,035.

The incubation steps of the above method may be accomplished by maintaining the ES cell and EB cultures in an environment wherein temperature and atmosphere are controlled. In one embodiment, the incubation condition is 37° C. in 6% CO₂.

The medium in the culture vessel may be selected from a variety of media known in the art that are suitable for maintaining ES cells in an undifferentiated state. In some embodiments, the medium comprises a basal medium and at least one differentiation-inhibiting factor. In one embodiment, the basal medium is DMEM. In one embodiment, the differentiation-inhibiting factor is mLIF. Once the formation of EB are desired, the ES cells may be removed to a medium lacking any differentiation-inhibiting factor, or the inhibiting factor may be neutralized with a suitable compound, so that differentiation may progress.

The medium of a composition and volume suitable for inducing EB formation and for culturing EB may be selected from a variety of media known in the art, or, alternatively, may be developed expressly for the purpose of inducing embryoid body formation from the particular cell culture at hand using well-known techniques of media development. The volume of medium may be selected as appropriate for the desired scale of the EB culture. In some embodiments, the volume of said medium is in the range of about 10 mL to about 10 L of medium. A general discussion of media preparation may be found in Freshney, R. I., Culture of Animal Cells: A Manual of Basic Technique, 4th Edition, Wiley-Liss (2000). Suitable media include natural media based on tissue extracts and bodily fluids as well chemically defined media. Media suitable for use with the present invention include media containing serum as well as media that is serum-free. Serum may be from any source, including calf, fetal bovine, horse, and human serum. Any selected medium may contain one or more of the following in any suitable combination: basal media, water, buffers, free-radical scavengers, detergents, surfactants, polymers, cellulose, salts, amino acids, vitamins, carbon sources, organic supplements, hormones, growth factors, antibiotics, nutrients and metabolites, lipids, minerals, and inhibitors. Media may be selected or developed so that a particular pH, CO₂ tension, oxygen tension, osmolality, viscosity, and/or surface tension results from the composition of the medium.

There are a large number of tissue culture media that exist for culturing tissue from animals. Exemplary basal media that may be also used to prepare a tissue culture medium of the present invention include Eagle's medium, Medium 199, CMRL 1066, MB 752/1, RPMI 1640, DMEM, and F12. In some embodiments, ES cells may be maintained in a basal medium, such as Dulbecco's Minimal Essential Media (DMEM), in order to effect more precise control over the activation of certain progenitor populations in the explant. In one embodiment, the stem cells are cultured in DMEM cell culture medium with about 10% to about 15% FBS. In another embodiment, the stem cells are cultured in DMEM with about 5% FBS. Moreover, the explants can be maintained in the absence of sera for extended periods of time. In certain embodiments of the invention, the growth factors or other mitogenic agents are not included in the primary media for maintenance of the cultures in vitro, but are used subsequently to cause proliferation of distinct populations of progenitor cells.

In describing various media herein, it should be understood that media reagents that are measured in terms of volume should be measured at room temperature and atmospheric pressure unless otherwise specified. For convenience, and in accord with the conventions of the art, media are described in terms of the components that are mixed together to make the medium. Interactions between components, as well as dissociations and reassociations of components, are not taken into account. For example, a medium may be said to contain 50 mM potassium chloride, even if, after the salt is mixed into the medium, the medium actually contains 50 mM potassium ion and 50 mM chloride ion and essentially none of the original undissociated salt.

In certain embodiments, a medium to be employed in a method disclosed herein comprises one or more buffering salts in addition to whatever buffering capacity may be provided by any complex biological extracts. In general, buffering salts are an acid or base having a pKa near (optionally within 1-2 pH units) of the desired pH of the medium. Exemplary buffing salts include phosphate salts (e.g., sodium phosphate, potassium phosphate), Tris (usually as a halide salt, such as Tris-Cl), the Good buffers (e.g., HEPES, MES, etc.) and many amino acids that may be used as buffering salts. Buffering salts may be added to media as an acid or base and then, if necessary, adjusted to the desired pH by using a strong acid or base (e.g., hydrochloric acid, phosphoric acid, sulfuric acid, sodium hydroxide, potassium hydroxide, etc.). Alternatively, buffer salts may be added in the salt form, such as NaH₂PO₄ (monosodium phosphate), KH₂PO₄, K₂HPO₄, etc. It may be desirable to mix multiple salt and acid or base forms of a particular buffer salt system. The pH that the medium is adjusted to will determine the concentration of the various ionic and non-ionic forms of the buffer salts. For example, a potassium phosphate buffered aqueous medium will contain PO₄³⁻, HPO₄²⁻, H₂PO⁻, H₃PO₄ and K⁺ in varying concentrations depending on the pH. An exemplary buffer salt system comprises between about 50 and 300 mM potassium or sodium phosphate, optionally between about 100 and 200 mM potassium or sodium phosphate, and, as a further option, approximately 150 mM potassium or sodium phosphate. Exemplary salts include sodium, potassium, magnesium, calcium, chloride, sulfate, phosphate and bicarbonate.

In certain embodiments, a medium to be employed in a method disclosed herein comprises one or more carbon sources in addition to whatever carbon sources may be provided by any complex biological extracts. Exemplary types of carbon sources include simple sugars, complex sugars, alcohols, lipids and organic acids. Exemplary carbon sources include glucose, glutamine, galactose, and pyruvate.

In certain embodiments, a medium to be employed in a method disclosed herein comprises one or more magnesium sources in addition to whatever magnesium may be provided by any complex biological extracts. A magnesium source may be any magnesium-containing compound that is soluble in the medium and has little or no undesirable effect on the cell culture. Exemplary magnesium sources include magnesium sulfate (MgSO₄), magnesium chloride (MgCl₂), magnesium hydroxide (Mg(OH)₂, although this basic magnesium source may require balancing with an acid), etc.

In certain embodiments, a medium to be employed in a method disclosed herein comprises one or more metal sources in addition to whatever metals may be provided by any complex biological extracts, magnesium sources or buffering salts. The one or more metal sources may be referred to as a metal mix, whether the one or more metal sources are added directly to the medium or mixed with water to form a metal solution prior to addition to the medium. It may be desirable to include one or more of the following metals in a medium: cobalt (Co), manganese (Mn), copper (Cu), boron (B), molybdenum (Mo), zinc (Zn), iron (Fe), calcium (Ca), aluminum (Al) and nickel (Ni). Optionally, a medium comprises at least two, at least three, at least four, at least five, at least six, at least seven, at least eight, at least nine, or all ten of the foregoing metals in some form. Most metals occur in a variety of valence states. Preferably metals are used in the following valence states: cobalt(II), manganese(II), copper(II), molybdenum(VI), zinc(II), iron(II), calcium(II), aluminum(III) and nickel(II). Boron may be supplied as the borate oxyanion, (BO₃)³⁻. Mo(VI) may be supplied as the molybdate oxyanion, (MoO₄)²⁻. Optionally, a medium comprises at least two, at least three, at least four, at least five, at least six, at least seven, at least eight, at least nine, or all ten of the foregoing metals in the preferred valence state. When not supplied as oxyanions (or other anionic molecular forms), the metals may be supplied as salts with one or more negatively charged counter ions, such as hydroxyl (OH)⁻, sulfate (SO₄)²⁻, and chloride Cl⁻. Metals supplied as oxyanions are generally supplied as salts with one or more positively charged counter ion, such as hydrogen H⁺, lithium Li⁺, sodium Na⁺, potassium K⁺, and ammonium (NH₄)⁺. Exemplary metal sources include CoCl₂, MnSO₄, CuCl₂, H₃BO₄, Na₂MoO₄, ZnSO₄, FeSO₄, CaCl₂, AlCl₃ and NiCl₂. The metal source molecule may also be a hydrate with one or more water molecules, such as, for example: CoCl₂-6H₂O, MnSO₄-5H₂O, CuCl₂-2H₂O, Na₂MoO₄-2H₂O, ZnSO₄-7H₂O, FeSO₄-7H₂O, CaCl₂-2H₂O, AlCl₃-6H₂O and NiCl₂-6H₂O. In certain embodiments, a metal mix may be prepared as a dry mix or a concentrate. A dry mix or concentrate will have proportional amounts of each metal such that upon reconstitution in water or upon addition to media, the appropriate concentration of each metal is obtained. Concentrates are often referred to in terms of the amount of dilution that is recommended to achieve the intended metal concentrations. For example, a 100× metal concentrate has a concentration of each metal that is 100 times higher than the concentration that is intended for use in cell culture. By mixing 1 mL of 100× metal concentrate with 99 mL of medium, the desired metal concentration is obtained.

In certain embodiments, a medium to be employed in a method disclosed herein comprises one or more vitamins or organic supplements in addition to whatever vitamins or organic supplements may be provided by any complex biological extracts. Exemplary vitamins that may be included in a medium are: p-aminobenzoic acid, L-ascorbic acid, biotin, D-pantothenate, choline, folic acid, myoinositol, nicotinamide, pyridoxine, riboflavin, thiamine, vitamin A, vitamin B12 and vitamin D. Most vitamins are weak acids or bases (or may have both acidic and basic moieties) and may be supplied in the acidic or basic form or with a suitable counterion. Exemplary organic supplements include amino acids, proteins, peptides, nucleosides, and metabolic intermediates. Exemplary amino acids include the essential amino acids and nonessential amino acids. For example, choline may be supplied as a salt with an anion such as chloride, pantothenate may be supplied as D-calcium pantothenate, and pyridoxine may be supplied as pyridoxine-HCl. Exemplary media comprise one, two, three, four, five, six, seven, eight, nine, ten, eleven, twelve, thirteen, or fourteen of the preceding vitamins. Suitable concentrated mixtures of vitamins are commercially available, such as Kao and Michayluk vitamin solution 100× (K3129, Sigma-Aldrich, St. Louis, Mo.).

Exemplary free-radical scavengers which may comprise the media of the invention include glutathione, 2-mercaptoethanol (beta-mercaptoethanol), monothioglycerol, and dithiothreitol. Exemplary antibiotics which may comprise the media of the invention include gentamycin, amphotericin B, ampicillin, ciporfloxacin, erythromycin, kanamycin, MRA, neomycin, nystatin, penicillin, polymixin B, streptomycin, teracyclin, and tylosin.

Growth factors contained within the media used in the present invention include any molecule that stimulates cell proliferation and differentiation. In certain embodiments, the culture is contacted with a growth factor. Examples of growth factors are mitogenic growth factors, e.g., the growth factor is selected from a group consisting of PDGF, LIF, IGF, TGF, FGF, EGF, HGF, hedgehog or VEGF. Another example of growth factors are members of the TGFβ superfamily, e.g., the DVR (dpp and vg1 related) family, e.g., BMP2 and/or BMP7. Growth factors may also be steroid or corticosteroid such as, for example, hydrocortisone, deoxyhydrocortisone, fludrocortisone, prednisolone, methylprednisolone, prednisone, triamcinolone, dexamethasone, betamethasone and paramethasone. See, generally, The Merck Manual of Diagnosis and Therapy, 15th Ed., pp. 1239-1267 and 2497-2506, Berkow et al., eds., Rahay, N.J. (1987).

Media described herein may be prepared as a dry mix or a concentrate. A dry mix will generally contain less than about 20% H₂O by weight, and may be in a powder or other solid or semisolid form. Some components, such as glycerol, tend to be liquids even in the absence of water. Media may also be prepared as a concentrate, such as a 100×, 20×, 10×, 5×, or 2× concentrate. Other components may be incorporated as needed.

Undifferentiated ES cells may be obtained from any mammalian source, including, but not limited to murines, canines, felines, bovines, equines, and primates. Such cells may be produced, stored, cultured, subcultured, and changed into different medium prior to use with the methods of the present invention for inducing embryoid body formation using methods for culturing and maintaining ES cells in an undifferentiated state as are well-known in the art. For a review of such methods, please see Roach and McNeish, Methods in Molecular Biology, 184:1-16, Humana Press (2002).

The methods of the present invention may be adapted for use with any undifferentiated embryonic or adult stem cell. In some embodiments, the undifferentiated embryonic stem cells are of mammalian origin. In one embodiment, the undifferentiated embryonic stem cells are of murine origin. In one embodiment, the undifferentiated embryonic stem cells are of human origin. In embodiments where the undifferentiated cell is an adult cell, the EB or EB equivalents are generally known in the art instead as “cellular aggregates” (for example a “neurosphere” results from the production of neural cells from adult undifferentiated neural progenitor cells).

3. Cellular Compositions

Another aspect of the invention relates to cellular compositions having EB with improved characteristics, such as EBs comprising cells with a particular phenotype. For example, EBs and the cells comprising them may be characterized by cell surface marker expression, size, glucose consumption, lactate production, and cell yield/viability from dissociated EBs. Methods for assessing the quality of an EB cellular composition are known in the art, see, e.g., Dang, et al., Biotech. Bioeng., 78:442-53 (2002), Sauer, et al., FEBS Lett., 476:218-23 (2000); and Magyar, et al., Ann. N.Y. Acad. Sci., 944:135-43 (2001).

In one embodiment, the present invention provides cellular compositions of EBs wherein a majority of the cells comprising the EBs have a particular phenotype. In certain embodiments, at least about 80%, 90% or 95% of the cells comprising the embryoid bodies have a diameter of at least about 11 microns. In certain embodiments, at least about 80%, 90% or 95% of the cells comprising the embryoid bodies have a diameter of at least about 12 microns. In certain embodiments, at least about 80%, 90% or 95% of the cell comprising the embryoid bodies have a diameter of at least about 13 microns. In certain embodiments, at least about 80%, 90% or 95% of the cells comprising the embryoid bodies have a diameter of at least about 14 microns. In other embodiments, at least 80%, 90% or 95% of the cells comprising the embryoid bodies have a diameter in the range of about 13 microns to about 15 microns. In certain embodiments, at least about 9%, 10%, 11%, 12%, or 14% of the cells comprising the embryoid bodies express CD34. In certain embodiments, at least about 20%, 25%, or 30% of the cells comprising the embryoid bodies express FLK-1.

In another embodiment, the present invention provides cellular compositions wherein the EBs are comprised of cells with high viability, for example, greater than 95%. In still other embodiments, the EB cellular compositions when dissociated may yield cells wherein at least about 94%, preferably at least about 95%, more preferably at least about 96%, 97%, 98% or 99% of the total cell population are viable.

In certain embodiments, the EB are produced through the above-described suspension methods. In other embodiments, EB of uniform size or morphology are obtained through physical methods. For example, a culture of EB may be passed through a physical sieve to select only the EB of a desired size and morphology. In another embodiment, a culture of EB may be subjected to gradient centrifugation to separate EB of a desired size and morphology. In still another embodiment, EB cells expressing certain biomarkers (e.g., cell surface protein) may be separated on the basis of the expression of the biomarkers. In preferred embodiments, the EB cells may be separated using a fluorescence activated cell sorter (FACS) or using magnetic beads.

In certain embodiments, the EBs or progeny thereof will be provided as part of a pharmaceutical preparation, e.g., sterile, free of the presence of unwanted virus, bacteria and other (human) pathogens, as well as pyrogen-free preparation. That is, for human administration, the subject EB or progeny preparations should meet sterility, pyrogenicity, general safety and purity standards as required by FDA Office of Biologics standards.

In certain embodiments, such cellular compositions can be used for transplantation into animals, preferably mammals, and even more preferably humans. The cells can be autologous, allogeneic or xenogeneic with respect to the transplantation host. In one aspect, the present invention relates to transplantation of fetal or mature pancreatic cells to treat Type 1 diabetes mellitus.

Yet another aspect of the present invention concerns cellular compositions which include, as a cellular component, substantially pure preparations of the subject progenitor cells, or the progeny thereof. Cellular compositions of the present invention include not only substantially pure populations of the progenitor cells, but can also include cell culture components, e.g., culture media including amino acids, metals, coenzyme factors, as well as small populations of non-progenitor cells, e.g., some of which may arise by subsequent differentiation of isolated progenitor cells of the invention. Furthermore, other non-cellular components include those which render the cellular component suitable for support under particular circumstances, e.g., implantation, e.g., continuous culture.

4. Large-Scale In Vitro Differentiation of EB into Other Cell Lineages and Tissues

The present invention also provides methods for large-scale in vitro differentiation using the above-described methods for producing uniform EB cultures. In one embodiment, the method for differentiating ES cells comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating the resulting embryoid bodies, inoculating the dissociated embryoid bodies into a medium suitable for accomplishing further differentiation; and culturing said dissociated embryoid bodies to a later differentiation state.

Cultures of EB of controlled size and morphology obtained through physical methods may also be incorporated into the subject large-scale in vitro differentiation methods. For example, a culture of EB may be passed through a physical sieve to select only the EB of a desired size and morphology. In another embodiment, a culture of EB may be subjected to gradient centrifugation to separate EB of a desired size and morphology. In yet another embodiment, the EB cells may be separated on the basis of expression of molecules on the cell surface, e.g., by FACS. Cultures of such EB may be dissociated, inoculated into a medium suitable for accomplishing further differentiation, and cultured to a later differentiation state.

In some embodiments, the dissociating step comprises trypsinization of the embryoid bodies. In some embodiments, the medium suitable for accomplishing further differentiation is comprised of at least one molecule that accomplishes final differentiation of said embryoid bodies, as known in the art.

The culturing step may be achieved by any of a variety of methods and may result in a variety of types of differentiated ES cells. In one embodiment, the culturing step results in the formation of a differentiated embryonic stem cell. In some embodiments, the differentiated stem cell may be any of macrophages, neural cells, pancreatic cells, cardiac cells, and the like. In some embodiments, the culturing step is accomplished at a large scale.

Preferably, the embryoid bodies are dissociated before continuing further differentiation. For example, dissociation may be achieved by trypsinization. Trypsinization generally comprises removing the media in which the EB have grown, washing the cultured EBs with a wash solution, such as PBS, then suspending the cells in a solution comprising trypsin. In certain embodiments of the present invention, the solution comprises 0.05% trypsin and EDTA. Because trypsinization may be damaging to the EB, other enzymes may be used alone or in combination in solutions to effect dissociation of the embryoid bodies. Such enzymes include, but are not limited to, collagenase, pronase, dispase, hyaluronidase, and neuraminidase. Alternatively, dissociation may be achieved by mechanical means, such as sieving or centrifugation, as known in the art.

Once the EB are dissociated, they generally are transferred into media suitable for achieving further differentiation.

Media for use with such techniques are well-known in the art, as are techniques for developing new media, as discussed for the media in section 4. Such media may comprise one or more growth factors to induce differentiation of the EB. Certain methods described herein employ polypeptide growth factors. Preparations of each of these factors are commercially available, and sources are listed in the Examples. It is also understood that one may employ variants, fragments and functional mimics, so long as such compounds can be provided at a concentration sufficient to provide similar functional activity.

Growth factors that induce final differentiation states are well-known in the art, and may be selected from any such factor that has been shown to induce a final differentiation state. Growth factors for use in methods described herein may, in certain embodiments, be variants or fragments of a naturally-occurring growth factor. For example, a variant may be generated by making conservative amino acid changes and testing the resulting variant in one of the functional assays described herein or another functional assay known in the art. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As those skilled in the art will appreciate, variants or fragments of polypeptide growth factors can be generated using conventional techniques, such as mutagenesis, including creating discrete point mutation(s), or by truncation. For instance, mutation can give rise to variants which retain substantially the same, or merely a subset, of the biological activity of a polypeptide growth factor from which it was derived.

Growth factor variants may also be chemically modified by forming covalent or aggregate conjugates with other chemical moieties, such as glycosyl groups, lipids, phosphate, acetyl groups and the like. Covalent derivatives can be prepared by linking the chemical moieties to functional groups on amino acid sidechains of the protein or at the N-terminus or at the C-terminus of the polypeptide.

Functional mimics of a growth factor include any compound that has an effect on at least a portion of the cellular signaling pathway of the relevant growth factor and is able to elicit a similar response in a functional assay for the growth factor, such as in one of the assays disclosed herein. As with fragments and variants, a functional mimic need not have the same concentration range for effectiveness, so long as the functional mimic is sufficiently active and non-toxic that there exists a practical concentration at which it can be used. A functional mimic may be generated by, for example, designing a molecule that activates the growth factor receptor, i.e., an EGF functional mimic could be a molecule that activates the EGF receptor.

In addition to the appropriate growth factors, other media components may be selected as appropriate for the cellular starting material, and some degree of routine optimization is expected for each culture situation. For example, commonly used media bases include Dulbecco's Modified Eagle's Medium (DMEM), Ham's F-12 nutrient mixture, Iscove's Modified Dulbecco's Medium (IMDM), McCoy's 5A, RPMI 1640, etc. Generally, differences between the different media can be compensated for with the addition or omission of supplements, such as carbon sources (e.g., glucose, pyruvate, etc.), serum (e.g., fetal bovine serum), vitamins, amino acids, etc. Other media components that may be selected and optimized to match the desired culture conditions are antibiotics (e.g., aminoglycosides such as gentamycin, penicillins, etc.) amino acids (particularly glutamine) and reducing agents (e.g., thiols such as monothioglycerol). Exemplary media compositions are set forth in the Examples below.

Methods for accomplishing large-scale cell culture are known in the art for both suspension and monolayer cultures. In generate, in such an expanded culture procedure a commercial-sized bioreactor, such as the CELLMAX® QUAD cell culture system (Cellco, Inc., Germantown, Md.), is seeded with a primary culture of human pancreatic cells. The bioreactor is perfused with a suitable, complete growth medium supplemented with an appropriately effective concentration of mitogens, and as appropriate, cAMP elevating agents. The cells can then be harvested. Cells may be cryopreserved prior to use as described, for example, by Beattie et al., Transplantation, 56:1340 (1993).

In general, scale-up in suspension cultures involves primarily an increase in the volume of the cultures. Such an increase in the volume may be achieved through the use of large stirrer or spinner flasks, large shaker flasks, fermentors, and bioreactors. Capacities of such vessels may range from 1 L to 20,000 L, depending on how large-scale the culture will be. Agitation of the medium may be necessary in such cultures, as may be sparging with air and other gases to maintain gas exchange. The progress of suspension cultures may be monitored, for example, via pH, oxygen, CO₂, and glucose electrodes that read from the culture in situ.

In general, scale-up in monolayer cultures involves increasing the surface area of the substrate in proportion to the number of cells and the volume of the medium. One such system for scaling up monolayer cultures is the Nunclon® Cell Factory. The Cell Factory is comprised of multiple Petri-dish-like units interconnected at two adjacent corners by vertical tubes. The tubes contain openings that allow medium to flow between the compartments when the unit is placed on its end, and still allow connection of a gas phase. A cell suspension in medium is prepared and is run into the chambers of the unit. The unit is laid flat and connected to a gas line. A single tray is often used as a pilot culture and is incubated along with the other trays as a control. Other systems for scaling-up monolayer cultures include multiarray disks, spirals, tubes, roller bottle culture, microcarriers, and perfused monolayer cultures. In particular, perfusion and microcarrier techniques may be used wherein the cells are to be used in cell replacement or tissue transplant therapy.

A discussion of both suspension and monolayer culture techniques, as well as their scale-up, may be found in Freshney, R. I., Culture of Animal Cells: A Manual of Basic Technique, 4th Edition, Wiley-Liss (2000).

Differentiated cell types that may be produced using these methods include, but are not limited to, macrophages, hepatocytes, pancreatic cells, neural cells, cardiac cells, smooth muscle cells. pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, tonsil tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, fetal tissue and umbilical tissue. In certain embodiments, the tissue is a non-neuronal animal tissue which does not include brain or central nervous system tissue.

In some embodiments, such as in embodiments effecting differentiation of macrophages, the progenitor cell population within the animal cell suspension is then allowed to proliferate in the presence of the growth factor population and takes on a non-adherent, floating characteristic. In certain instances, the progenitor cell population forms homotypic cell spheres. The phenotypic characteristics of the progenitor cell population provide both an indication that the cell suspension has become enriched in the stem/progenitor cell population as well as providing certain physical features which may be used to enrich for the stem/progenitor cells.

Such cells may be further induced to growth into tissues using methods known in the art.

The above methods can be incorporated into a variety of methodologies comprising the large-scale in vitro differentiation of ES cells, including, but not limited to, screening methods, methods for producing tissues for transplantation or producing cells for cell replacement therapy, gene expression profiling, and gene cloning.

5. Methods of Using Cellular Compositions and Other Cells Produced by the Present Method

5. a. Compound Screening

The methods and cellular compositions of the present invention may be used in methods of screening candidate compounds using differentiated ES cells. In one embodiment, a method of screening compounds using differentiated embryonic stem cells comprises the steps of inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating said embryoid bodies, culturing said dissociated embryoid bodies to a later differentiation state, and contacting a differentiated cell produced by said culturing step with a therapeutic agent. In another embodiment, a method of screening compounds using differentiated embryonic stem cells comprises the steps of (a) producing EB of a uniform phenotype; (b) dissociating the embryoid bodies; (c) inoculating the dissociated embryoid bodies into a medium suitable for accomplishing further differentiation; (d) culturing said dissociated embryoid bodies to a later differentiation state; and (e) contacting a differentiated cell produced by said culturing step with a therapeutic agent. In another embodiment, a method of screening compounds using differentiated embryonic stem cells comprises contacting a cellular composition of the present invention with a compound

Assays and methods of developing assays appropriate for use in the methods described above are known to those of skill in the art, and are contemplated for use as appropriate with the methods of the present invention. The ability of said compound to interact with a ES cell of the present invention or have an effect on an ES cell or cellular composition of the present invention may be determined using a variety of appropriate assays known to those of skill in the art. In certain embodiments of the present invention, a candidate compound may be evaluated by an in vitro assay. In certain embodiments, the assay may be an in vivo assay. Assays may be conducted to identify molecules that modulate the expression and/or activity of a gene. Alternatively, assays may be conducted to identify molecules that modulate the activity of a protein encoded by a gene. Such assays are well-known to one of skill in the art and may be adapted to the methods of the present invention with no more than routine experimentation.

Compounds for use with the present invention may be selected from any of lipids, carbohydrates, peptides, peptidomimetics, peptide-nucleic acids (PNAs), proteins, small molecules, natural products, aptamers and oligonucleotides. Such compounds may be selected from a library of such compounds. The synthesis and screening of combinatorial libraries is a validated strategy for the identification and study of compounds of interest. According to the present invention, the synthesis of libraries containing molecules or compounds may be performed using established combinatorial methods for solution phase, solid phase, or a combination of solution phase and solid phase synthesis techniques. The synthesis of combinatorial libraries is well known in the art and has been reviewed (see, e.g., “Combinatorial Chemistry”, Chemical and Engineering News, p. 43 (Feb. 24, 1997); Thompson et al., Chem. Rev., 96:555 (1996)). Many libraries are commercially available. One of ordinary skill in the art will realize that the choice of method for any particular embodiment will depend upon the specific number of molecules to be synthesized, the specific reaction chemistry, and the availability of specific instrumentation, such as robotic instrumentation for the preparation and analysis of the inventive libraries. In certain embodiments, the reactions to be performed to generate the libraries are selected for their ability to proceed in high yield, and in a stereoselective and regioselective fashion, if applicable.

All of the above screening methods may be accomplished using a variety of assay formats. In light of the present disclosure, those not expressly described herein will nevertheless be known and comprehended by one of ordinary skill in the art. The assays may identify compounds which are, e.g., either agonists or antagonists of expression of a target gene of interest, or of a protein-protein or protein-substrate interaction of a target of interest, or of the role of target gene products in the pathogenesis of normal or abnormal cellular physiology, proliferation, and/or differentiation and disorders related thereto. The assays may further identify compounds which affect the generation of normal or abnormal cellular physiology, cell proliferation, and/or cell differentiation and disorders related thereto. Assay formats which approximate such conditions as formation of protein complexes or protein-nucleic acid complexes, enzymatic activity, and even specific signaling pathways in ES cells, may be generated in many different forms, and include but are not limited to assays based on cell-free systems, e.g., purified proteins or cell lysates, as well as cell-based assays which utilize intact cells.

5.a. 1. In Vivo Methods

One aspect of the present invention provides methods for screening various compounds for their ability to modulate growth, proliferation or differentiation of distinct progenitor cell populations from ES cell culture. Such methods are referred to within this section as in vivo as they involve the use of whole ES cells in culture. In an illustrative embodiment, the subject progenitor cells, and their progeny, can be used to screen various compounds. Such cells can be maintained in minimal culture media for extended periods of time (e.g., for 7-21 days or longer) and can be contacted with any compound, to determine the effect of such compound on one of cellular growth, proliferation or differentiation of progenitor cells in the culture. Detection and quantification of growth, proliferation or differentiation of these cells in response to a given compound provides a means for determining the compound's efficacy at inducing one of the growth, proliferation or differentiation in a given ductal explant. Methods of measuring cell proliferation are well known in the art and most commonly include determining DNA synthesis characteristic of cell replication. There are numerous methods in the art for measuring DNA synthesis, any of which may be used according to the invention. In an embodiment of the invention, DNA synthesis has been determined using a radioactive label (³H-thymidine) or labeled nucleotide analogues (BrdU) for detection by immunofluorescence. The efficacy of the compound can be assessed by generating dose response curves from data obtained using various concentrations of the compound. A control assay can also be performed to provide a baseline for comparison. Identification of the progenitor cell population(s) amplified in response to a given test compound can be carried out according to such phenotyping as described above.

In still further embodiments, a protein-protein, protein-substrate, or protein-nucleic acid interaction of interest is generated in whole cells, taking advantage of cell culture techniques to support the subject assay. For example, as described below, the interaction of interest may be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the compound to gain entry into the cell. Furthermore, certain of the in vivo embodiments of the assay, such as examples given below, are amenable to high through-put analysis of candidate agents.

The components of the interaction of interest may be endogenous to the cell selected to support the assay. Alternatively, some or all of the components may be derived from exogenous sources. For instance, fusion proteins may be introduced into the cell by recombinant techniques (such as through the use of an expression vector), as well as by microinjecting the fusion protein itself or mRNA encoding the fusion protein.

In any case, the cell may be ultimately manipulated after incubation with a candidate inhibitor in order to facilitate detection of a protein-protein, protein-substrate, or protein-nucleic acid interaction-mediated signaling event (e.g., modulation of a post-translational modification of a protein-protein interaction component substrate, such as phosphorylation, modulation of transcription of a gene in response to cell signaling, etc.). As described above for assays performed in reconstituted protein mixtures or lysate, the effectiveness of a candidate inhibitor may be assessed by measuring direct characteristics of an interaction component, such as shifts in molecular weight by electrophoretic means or detection in a binding assay. For these embodiments, the cell will typically be lysed at the end of incubation with the candidate agent, and the lysate manipulated in a detection step in much the same manner as might be the reconstituted protein mixture or lysate, e.g., described above.

Indirect measurement of an interaction may also be accomplished, for example, by detecting a biological activity associated with a protein-protein interaction component that is modulated by a protein-protein interaction mediated signaling event. As set out above, the use of fusion proteins comprising a protein-protein interaction component polypeptide and an enzymatic activity are representative embodiments of the subject assay in which the detection means relies on indirect measurement of a protein-protein interaction component polypeptide by quantitating an associated enzymatic activity.

In other embodiments, the biological activity of a component polypeptide taking part in a nucleic acid-protein, protein-substrate or protein-protein interaction may be assessed by monitoring changes in the phenotype of the targeted cell. For example, the detection means may include a reporter gene construct which includes a transcriptional regulatory element that is dependent in some form on the level of an interaction component or a interaction component substrate. The protein interaction component may be provided as a fusion protein with a domain which binds to a DNA element of the reporter gene construct. The added domain of the fusion protein may be one which, through its DNA-binding ability, increases or decreases transcription of the reporter gene. Whichever the case may be, its presence in the fusion protein renders it responsive to the protein-protein interaction-mediated signaling pathway. Accordingly, the level of expression of the reporter gene will vary with the level of expression of the protein interaction component.

The reporter gene product is preferably a detectable label, such as luciferase, β-lactamase or β-galactosidase, and is produced in the intact cell. The label may be measured in a subsequent lysate of the cell. However, the lysis step is preferably avoided, and providing a step of lysing the cell to measure the label will typically only be employed where detection of the label cannot be accomplished in whole cells.

Moreover, in the whole cell embodiments of the subject assay, the reporter gene construct may provide, upon expression, a selectable marker. A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. For instance, the product of the reporter gene may be an enzyme which confers resistance to antibiotic or other drug, or an enzyme which complements a deficiency in the host cell (i.e. thymidine kinase or dihydrofolate reductase). To illustrate, the aminoglycoside phosphotransferase encoded by the bacterial transposon gene Tn5 neo may be placed under transcriptional control of a promoter element responsive to the level of a protein-protein interaction component polypeptide present in the cell. Such embodiments of the subject assay are particularly amenable to high through-put analysis in that proliferation of the cell may provide a simple measure of inhibition of an interaction.

Other examples of reporter genes include, but are not limited to CAT (chloramphenicol acetyl transferase) (Alton and Vapnek, Nature, 282: 864-69 (1979)) luciferase, and other enzyme detection systems, such as β-galactosidase, β-lactamase, (G. Zlokamik, et al., Science, 279:84-88 (1998)); firefly luciferase (deWet et al., Mol. Cell. Biol., 7:725-37 (1987)); bacterial luciferase (Engebrecht and Silverman, , PNAS, 1: 4154-58 (1984); Baldwin et al., Biochemistry, 23: 3663-67 (1984)); alkaline phosphatase (Toh et al., Eur. J. Biochem., 182:231-38 (1989); Hall et al., J. Mol. Appl. Gen., 2:101 (1983)), human placental secreted alkaline phosphatase (Cullen and Malim, Methods in Enzymol., 216:362-68 (1992)).

The amount of transcription from the reporter gene may be measured using any method known to those of skill in the art to be suitable. For example, specific mRNA expression may be detected using Northern blots or specific protein product may be identified by a characteristic stain, western blots or an intrinsic activity.

In preferred embodiments, the product of the reporter gene is detected by an intrinsic activity associated with that product. For instance, the reporter gene may encode a gene product that, by enzymatic activity, gives rise to a detection signal based on color, fluorescence, or luminescence.

The amount of expression from the reporter gene is then compared to the amount of expression in either the same cell in the absence of the test compound or it may be compared with the amount of transcription in a substantially identical cell that lacks a component of the interaction of interest.

5.a.2. In Vitro Methods

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they may be generated to permit rapid development and relatively easy detection of an alteration in a molecular target which is mediated by a test compound. Moreover, the effects of cellular toxicity and/or bioavailability of the test compound may be generally ignored in the in vitro system, the assay instead being focused primarily on the effect of the compound on the molecular target as may be manifest in an alteration of binding affinity with other proteins or changes in enzymatic properties of the molecular target. Accordingly, potential modifiers, e.g., activators or inhibitors of protein-substrate, protein-protein interactions or nucleic acid-protein interactions of interest may be detected in a cell-free assay generated by constitution of function interactions of interest in a cell lysate. Such protein-substrate, protein-protein, or nucleic acid-protein interactions of interest may be identified by the gene expression profiling methods described herein. In an alternate format, the assay may be derived as a reconstituted protein mixture which, as described below, offers a number of benefits over lysate-based assays.

In one aspect, the present invention provides assays that may be used to screen for compounds which modulate protein-protein interactions, nucleic acid-protein interactions, or protein-substrate interactions of interest. For instance, the drug screening assays of the present invention may be designed to detect agents which disrupt binding of protein-protein interaction binding moieties. In other embodiments, the subject assays will identify inhibitors of the enzymatic activity of a protein or protein-protein interaction complex. In a preferred embodiment, the compound is a mechanism based inhibitor which chemically alters one member of a protein-protein interaction or one chemical group of a protein and which is a specific inhibitor of that member, e.g., has an inhibition constant 10-fold, 100-fold, or more preferably, 1000-fold different compared to homologous proteins.

In one embodiment of the present invention, screening assays may be generated which detect inhibitory compounds on the basis of their ability to interfere with binding of components of a given protein-substrate, protein-protein, or nucleic acid-protein interaction of interest. In an exemplary binding assay, the compound of interest is contacted with a mixture generated from protein-protein interaction component polypeptides. Detection and quantification of expected activity from a given protein-protein interaction provides a means for determining the compound's efficacy at inhibiting (or potentiating) complex formation between the two polypeptides. The efficacy of the compound may be assessed by generating dose response curves from data obtained using various concentrations of the test compound. Moreover, a control assay may also be performed to provide a baseline for comparison. In the control assay, the formation of complexes is quantitated in the absence of the test compound.

Complex formation between component polypeptides, polypeptides and genes, or between a component polypeptide and a substrate may be detected by a variety of techniques, many of which are effectively described above. For instance, modulation in the formation of complexes may be quantitated using, for example, detectably labeled proteins (e.g., radiolabeled, fluorescently labeled, or enzymatically labeled), by immunoassay, or by chromatographic detection.

Accordingly, one exemplary screening assay of the present invention includes the steps of contacting a polypeptide or functional fragment thereof or a binding partner with a test compound or library of test compounds and detecting the formation of complexes. For detection purposes, the molecule may be labeled with a specific marker and the test compound or library of test compounds labeled with a different marker. Interaction of a test compound with a polypeptide or fragment thereof or binding partner may then be detected by determining the level of the two labels after an incubation step and a washing step. The presence of two labels after the washing step is indicative of an interaction.

An interaction between molecules may also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds may be immobilized on a sensor surface, e.g., which forms one wall of a micro-flow cell. A solution containing the polypeptide, functional fragment thereof, polypeptide analog or binding partner is then flowed continuously over the sensor surface. A change in the resonance angle, as shown on a signal recording, indicates that an interaction has occurred. This technique is further described, e.g., in BIAtechnology Handbook by Pharmacia.

Another exemplary screening assay of the present invention includes the steps of forming a reaction mixture including a polypeptide, a binding partner, and a test compound, and detecting interaction of the polypeptide and the binding partner. The polypeptide and binding partner may be produced recombinantly, purified from a source, e.g., plasma, or chemically synthesized, as described herein. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of polypeptide bioactivity for the test compound. The compounds of this assay may be contacted simultaneously. Alternatively, a polypeptide may first be contacted with a test compound for an appropriate amount of time, following which the binding partner is added to the reaction mixture. The efficacy of the compound may be assessed by generating dose response curves from data obtained using various concentrations of the test compound. Moreover, a control assay may also be performed to provide a baseline for comparison. In the control assay, isolated and purified polypeptide or binding partner is added to a composition containing the binding partner or polypeptide, and the formation of a complex is quantitated in the absence of the test compound.

Complex formation between a polypeptide and a binding partner may be detected by a variety of techniques. Modulation of the formation of complexes may be quantitated using, for example, detectably labeled proteins such as radiolabeled, fluorescently labeled, or enzymatically labeled polypeptides or binding partners, by immunoassay, or by chromatographic detection.

Typically, it will be desirable to immobilize either polypeptide or its binding partner to facilitate separation of complexes from uncomplexed forms of one or both of the proteins, as well as to accommodate automation of the assay. Binding of polypeptide to a binding partner, may be accomplished in any vessel suitable for containing the reactants. Examples include microtitre plates, test tubes, and micro-centrifuge tubes. In one embodiment, a fusion protein may be provided which adds a domain that allows the protein to be bound to a matrix. For example, glutathione-S-transferase/polypeptide (GST/polypeptide) fusion proteins may be adsorbed onto glutathione sepharose beads (Sigma Chemical, St. Louis, Mo.) or glutathione derivatized microtitre plates, which are then combined with the binding partner, e.g., an ³⁵S-labeled binding partner, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes may be dissociated from the matrix, separated by SDS-PAGE, and the level of polypeptide or binding partner found in the bead fraction quantitated from the gel using standard electrophoretic techniques such as described in the appended examples.

Other techniques for immobilizing proteins on matrices are also available for use in the subject assay. For instance, either the polypeptide or its cognate binding partner may be immobilized utilizing conjugation of biotin and streptavidin. For instance, biotinylated polypeptide molecules may be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptide may be derivatized to the wells of the plate, and polypeptide trapped in the wells by antibody conjugation. As above, preparations of a binding partner and a test compound are incubated in the polypeptide presenting wells of the plate, and the amount of complex trapped in the well may be quantitated. Exemplary methods for detecting such complexes, in addition to those described above for the GST-immobilized complexes, include immunodetection of complexes using antibodies reactive with the binding partner, or which are reactive with polypeptide and compete with the binding partner, as well as enzyme-linked assays which rely on detecting an intrinsic or extrinsic enzymatic activity associated with the binding partner. In the instance of the latter, the enzyme may be chemically conjugated or provided as a fusion protein with the binding partner. To illustrate, the binding partner may be chemically cross-linked or genetically fused with horseradish peroxidase, and the amount of polypeptide trapped in the complex may be assessed with a chromogenic substrate of the enzyme, e.g., 3,3′-diamino-benzadine tetrahydrochloride or 4-chloro-1-napthol. Likewise, a fusion protein comprising the polypeptide and glutathione-S-transferase may be provided, and complex formation quantitated by detecting the GST activity using 1-chloro-2,4-dinitrobenzene (Habig et al., J. Biol. Chem., 249:7130 (1974)).

For processes that rely on immunodetection for quantitating one of the proteins trapped in the complex, antibodies against the protein, such as anti-polypeptide antibodies, may be used. Alternatively, the protein to be detected in the complex may be “epitope-tagged” in the form of a fusion protein which includes, in addition to the polypeptide sequence, a second polypeptide for which antibodies are readily available (e.g., from commercial sources). For instance, the GST fusion proteins described above may also be used for quantification of binding using antibodies against the GST moiety. Other useful epitope tags include myc-epitopes (e.g., see Ellison et al., J. Biol. Chem., 266:21150-57 (1991)) which includes a 10-residue sequence from c-myc, as well as the pFLAG system (International Biotechnologies, Inc.) or the pEZZ-protein A system (Pharmacia, N.J.).

In certain in vitro embodiments of the present assay, the protein or the set of proteins engaged in a protein-protein, protein-substrate, or protein-nucleic acid interaction comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-substrate, protein-protein or nucleic acid-protein interaction are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-substrate, protein-protein interaction, or nucleic acid-protein interaction.

In one embodiment, the use of reconstituted protein mixtures allows more careful control of the protein-substrate, protein-protein, or nucleic acid-protein interaction conditions. Moreover, the system may be derived to favor discovery of inhibitors of particular intermediate states of the protein-protein interaction. For instance, a reconstituted protein assay may be carried out both in the presence and absence of a candidate agent, thereby allowing detection of an inhibitor of a given protein-substrate, protein-protein, or nucleic acid-protein interaction.

Assaying biological activity resulting from a given protein-substrate, protein-protein or nucleic acid-protein interaction, in the presence and absence of a candidate inhibitor, may be accomplished in any vessel suitable for containing the reactants. Examples include microtitre plates, test tubes, and micro-centrifuge tubes.

Typically, it will be desirable to immobilize one of the polypeptides to facilitate separation of complexes from uncomplexed forms of one of the proteins, as well as to accommodate automation of the assay. In an illustrative embodiment, a fusion protein may be provided which adds a domain that permits the protein to be bound to an insoluble matrix. For example, protein-protein interaction component fusion proteins may be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when a microtitre plate is used. Alternatively, after washing away unbound protein, the complexes may be dissociated from the matrix, separated by SDS-PAGE gel, and the level of interacting polypeptide found in the matrix-bound fraction quantitated from the gel using standard electrophoretic techniques.

In yet another embodiment, the protein-protein interaction component or potential interacting polypeptide may be used to generate an two-hybrid or interaction trap assay (see also, U.S. Pat. No. 5,283,317; Zervos et al., Cell, 72:223-32 (1993); Madura et al., J. Biol. Chem., 268:12046-54 (1993); Bartel et al., Biotechniques, 14:920-24 (1993); and Iwabuchi et al., Oncogene, 8:1693-96 (1993)), for subsequently detecting agents which disrupt binding of the interaction components to one another.

In particular, the method makes use of chimeric genes which express hybrid proteins. To illustrate, a first hybrid gene comprising the coding sequence for a DNA-binding domain of a transcriptional activator may be fused in frame to the coding sequence for a “bait” protein, e.g., a protein-protein interaction component polypeptide of sufficient length to bind to a potential interacting protein. The second hybrid protein encodes a transcriptional activation domain fused in frame to a gene encoding a “fish” protein, e.g., a potential interacting protein of sufficient length to interact with the protein-protein interaction component polypeptide portion of the bait fusion protein. If the bait and fish proteins are able to interact, e.g., form a protein-protein interaction component complex, they bring into close proximity the two domains of the transcriptional activator. This proximity causes transcription of a reporter gene which is operably linked to a transcriptional regulatory site responsive to the transcriptional activator, and expression of the reporter gene may be detected and used to score for the interaction of the bait and fish proteins.

In accordance with the present invention, the method includes providing a host cell. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a protein-protein interaction component polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other substantially transcriptionally-inert DNA-binding domains may be used in the subject constructs; such as domains of ACE1, λcl, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al., PCT Patent Application Publication WO 94/10300).

In preferred embodiments, any enzymatic activity associated with the bait or fish proteins is inactivated, e.g., dominant negative or other mutants of a protein-protein interaction component may be used.

Continuing with the illustrated example, the protein-protein interaction component-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a protein-protein interaction component/interacting protein complex results in a detectable signal produced by the expression of the reporter gene. Accordingly, the level of formation of a complex in the presence of a test compound and in the absence of the test compound may be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations, e.g., combinations of proteins participating in protein-protein interactions.

5.a.3. Efficacy Testing

The efficacy of the compounds may then be tested in additional in vitro assays and in vivo. A test compound may be administered to a cell or tissue produced by the methods of the invention and at least one characteristic or behavior of the tissue or cell monitored. Expression of one or more genes characteristic of a particular disorder, proliferative state, or differentiation state may also be measured before and after administration of the test compound to the animal. A normalization of the expression of one or more of these genes is indicative of the efficiency of the compound for treating disorders in the animal.

In another embodiment of the invention, a drug is developed by rational drug design, i.e., it is designed or identified based on information stored in computer readable form and analyzed by algorithms. More and more databases of expression profiles are currently being established, numerous ones being publicly available. The present invention provides expression profiles as well as methods for generating them (see next section). By screening such databases for the description of drugs affecting the expression of at least some of the genes characteristic of a disorder in a manner similar to the change in gene expression profile from a diseased ES cell to that of a normal ES cell corresponding to the diseased ES cell, compounds may be identified which normalize gene expression in a diseased ES cell. Derivatives and analogues of such compounds may then be synthesized to optimize the activity of the compound, and tested and optimized as described above.

Compounds identified by the methods described above are within the scope of the invention. Compositions comprising such compounds, in particular, compositions comprising a pharmaceutically efficient amount of the drug in a pharmaceutically acceptable carrier are also provided.

5.b. Gene Expression Profiling.

In part, the present invention is directed to the use of cells produced by the methods of the invention as well as cellular compositions of the invention to assess gene expression. In another part, the present invention is directed to the use of subject nucleic acids isolated from the cellular compositions and cells produced by methods of the present invention in arrays.

In one embodiment, a method of gene expression profiling comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating the resulting embryoid bodies, inoculating the dissociated embryoid bodies into a medium suitable for accomplishing further differentiation, culturing said dissociated embryoid bodies to a later differentiation state, and determining the gene expression in a differentiated cell produced by said culturing step. In another embodiment, a method of gene expression profiling comprises producing EB of a uniform phenotype, dissociating the resulting embryoid bodies, inoculating the dissociated embryoid bodies into a medium suitable for accomplishing further differentiation, culturing said dissociated embryoid bodies to a later differentiation state, and determining the gene expression in a differentiated cell produced by said culturing step. In other embodiments, a method of gene expression profiling comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, and determining the gene expression of a cell or embryoid body in said shaker flask. In other embodiments of the present invention, the protein activity of a cell or embryoid body will be determined rather than the gene expression.

A person of skill in the art will recognize that in certain screening assays, it will be sufficient to assess the level of expression of a single gene and that in others, the expression of two or more is preferred, whereas still in others, the expression of essentially all the genes involved in a particular cellular activity is preferably assessed. Likewise, it will be sufficient to assess the activity of a single protein in some screening assays, whereas in others, the activities of multiple proteins may be assessed. Such assays are well-known to one of skill in the art and may be adapted to the methods of the present invention with no more than routine experimentation.

In one aspect, the present invention provides an array comprising a substrate having a plurality of addresses, wherein at least one of the addresses has disposed thereon a capture probe that can specifically bind to a nucleic acid of the invention. In another aspect, the present invention contemplates a method for detecting expression of a nucleotide sequence which encodes a polypeptide, or a fragment thereof, using the foregoing array by: (a) providing a sample comprising at least one mRNA molecule; (b) exposing the sample to the array under conditions which promote hybridization between the capture probe disposed on the array and a nucleic acid complementary thereto; and (c) detecting hybridization between an mRNA molecule of the sample and the capture probe disposed on the array, thereby detecting expression of a sequence which encodes for a polypeptide, or a fragment thereof.

Arrays are often divided into microarrays and macroarrays, where microarrays have a much higher density of individual probe species per area. Microarrays may have as many as 1000 or more different probes in a 1 cm² area. There is no concrete cut-off to demarcate the difference between micro- and macroarrays, and both types of arrays are contemplated for use with the invention.

Microarrays are known in the art and generally consist of a surface to which probes that correspond in sequence to gene products (e.g., cDNAs, mRNAs, oligonucleotides) are bound at known positions. In one embodiment, the microarray is an array (e.g., a matrix) in which each position represents a discrete binding site for a product encoded by a gene (e.g., a protein or RNA), and in which binding sites are present for products of most or almost all of the genes in the organism's genome. In certain embodiments, the binding site is a nucleic acid or nucleic acid analogue to which a particular cognate cDNA can specifically hybridize. The nucleic acid or analogue of the binding site may be, e.g., a synthetic oligomer, a full-length cDNA, a less-than full length cDNA, or a gene fragment.

Although in certain embodiments the microarray contains binding sites for products of all or almost all genes in the target organism's genome, such comprehensiveness is not necessarily required. Usually the microarray will have binding sites corresponding to at least 100, 500, 1000, 4000 genes or more. In certain embodiments, arrays will have anyway from about 50, 60, 70 , 80, 90, even more than 95% of the genes of a particular organism represented. The microarray typically has binding sites for genes relevant to testing and confirming a biological network model of interest. Several exemplary human microarrays are publicly available.

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (e.g., fragments that do not share more than 10 bases of contiguous identical sequence with any other fragment on the microarray). Computer programs are useful in the design of primers with the required specificity and optimal amplification properties. See, e.g., Oligo pl version 5.0 (National Biosciences). In an alternative embodiment, the binding (hybridization) sites are made from plasmid or phage clones of genes, cDNAs (e.g., expressed sequence tags), or inserts therefrom (Nguyen et al., 1995, Genomics 29:207-209).

A number of methods are known in the art for affixing the nucleic acids or analogues to a solid support that makes up the array (Schena et al., Science, 270:467-70 (1995); DeRisi et al., Nature Genetics, 14:457-60 (1996); Shalon et al., Genome Res., 6:639-45 (1996); and Schena et al., PNAS, 93:10539-11286 (1995)).

Another method for making microarrays is by making high-density oligonucleotide arrays (Fodor et al., Science, 251:767-73 (1991); Pease et al., PNAS, 91:5022-26 (1994); Lockhart et al., Nature Biotech., 14:1675 (1996); U.S. Pat. Nos. 5,578,832; 5,556,752; and 5,510,270; Blanchard et al., Biosens. Bioelec., 11:687-90 (1996)).

Other methods for making microarrays, e.g., by masking (Maskos and Southern, Nuc. Acids Res., 20:1679-84 (1992)), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y. (1989)), could be used, as will be recognized by those of skill in the art.

The nucleic acids to be contacted with the microarray may be prepared in a variety of ways, and may include nucleotides of the subject invention. Such nucleic acids are often labeled fluorescently. Nucleic acid hybridization and wash conditions are chosen so that the population of labeled nucleic acids will specifically hybridize to appropriate, complementary nucleic acids affixed to the matrix. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array may be detected by scanning confocal laser microscopy. When two fluorophores are used, a separate scan, using the appropriate excitation line, is carried out for each of the two fluorophores used. Fluorescent microarray scanners are commercially available from Affymetrix, Packard BioChip Technologies, BioRobotics and many other suppliers. Signals are recorded, quantitated and analyzed using a variety of computer software.

The next step is to contact the labeled nucleic acids with the array under conditions sufficient for binding between the probe and the target of the array. In a preferred embodiment, the probe will be contacted with the array under conditions sufficient for hybridization to occur between the labeled nucleic acids and probes on the microarray, where the hybridization conditions will be selected in order to provide for the desired level of hybridization specificity.

Contact of the array and probe involves contacting the array with an aqueous medium comprising the probe. Contact may be achieved in a variety of different ways depending on specific configuration of the array. For example, where the array simply comprises the pattern of size separated targets on the surface of a “plate-like” rigid substrate, contact may be accomplished by simply placing the array in a container comprising the probe solution, such as a polyethylene bag, and the like. In other embodiments where the array is entrapped in a separation media bounded by two rigid plates, the opportunity exists to deliver the probe via electrophoretic means. Alternatively, where the array is incorporated into a biochip device having fluid entry and exit ports, the probe solution may be introduced into the chamber in which the pattern of target molecules is presented through the entry port, where fluid introduction could be performed manually or with an automated device. In multiwell embodiments, the probe solution will be introduced in the reaction chamber comprising the array, either manually, e.g., with a pipette, or with an automated fluid handling device.

Contact of the probe solution and the targets will be maintained for a sufficient period of time for binding between the probe and the target to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions may be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It may easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and may also be tested in assays. An extensive guide to the hybridization of nucleic acids is found in Tijssen (Laboratory Techniques in Biochemistry and Molecular Biology, Vol. 24: Hybridization With Nucleic Acid Probes, P. Tijssen, ed. Elsevier, N.Y., (1993)), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes may be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, may be quite small, corresponding to a difference in Tm between the two of as little as 0.5 degrees. See Tibanyenda et al., Eur. J. Biochem., 139:19 (1984) and Ebel et al., Biochem., 31:12083 (1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Laboratory Techniques in Biochemistry and Molecular Biology, Vol. 24: Hybridization With Nucleic Acid Probes, P. Tijssen, ed. Elsevier, N.Y., (1993), e.g., part I chapter 2 “Overview of principles of hybridization and the strategy of nucleic acid probe assays”, provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). The active devices of this invention may electronically produce “different stringency conditions” at each microlocation. Thus, all hybridizations may be carried out optimally in the same bulk solution. These arrays are described in U.S. Pat. No. 6,051,380 by Sosnowski et al.

In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target may be removed by electrophoretic means.

Where all of the target sequences are detected using the same label, different arrays will be employed for each physiological source (where different could include using the same array at different times). The above methods may be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device imaging camera (Guschin et al., Anal. Biochem., 250:203 (1997)). Synthesis of arrays on optical fibre bundles allows easy and sensitive reading (Healy et al., Anal. Biochem., 251:270 (1997)). In another embodiment, real time hybridization detection is carried out on microarrays without washing using evanescent wave effect that excites only fluorophores that are bound to the surface (see, e.g., Stimpson et al., PNAS, 92:6379 (1995)).

The above steps result in the production of hybridization patterns of labeled target nucleic acid on the array surface. The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, calorimetric measurement, light emission measurement, light scattering, and the like.

One method of detection includes an array scanner that is commercially available from Affymetrix (Santa Clara, Calif.), e.g., the 417™ Arrayer, the 418™ Array Scanner, or the Agilent GeneArray™ Scanner. This scanner is controlled from the system computer with a Windows® interface and easy-to-use software tools. The output is a 16-bit .tif file that may be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores may be analyzed simultaneously (see Shalon et al., Genome Research, 6:639-45 (1996)). In a preferred embodiment, the arrays are scanned with a laser fluorescent scanner with a computer controlled X-Y stage and a microscope objective. Sequential excitation of the two fluorophores may be achieved with a multi-line, mixed gas laser and the emitted light is split by wavelength and detected with two photomultiplier tubes. Fluorescence laser scanning devices are described in Schena et al., Genome Res., 6:639-45 (1996) and in other references cited herein. Alternatively, the fiber-optic bundle described by Ferguson et al., Nature Biotech., 14:1681-84 (1996), may be used to monitor mRNA abundance levels.

In one embodiment in which fluorescent target nucleic acids are used, the arrays may be scanned using lasers to excite fluorescently labeled targets that have hybridized to regions of probe arrays, which may then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a cell of a subject having a disorder and the expression profile of a counterpart normal cell in a subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively very efficient. The accuracy of such algorithms is preferably hierarchically adjustable so that the level of detail of clustering may be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, Statistical Pattern Recognition, 2nd Ed., Academic Press, San Diego (1990); Everitt, Cluster Analysis, London: Heinemann Educ. Books (1974); Hartigan, Clustering Algorithms, New York: Wiley (1975); Sneath and Sokal, Numerical Taxonomy, Freeman (1973); Anderberg, Cluster Analysis for Applications, Academic Press: New York (1973)).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g., U.S. Pat. No. 6,203,987). Other systems permit clustering of time curves (see, e.g., U.S. Pat. No. 6,263,287). Cluster analysis may be performed using the hclust routine (see, e.g., “hclust” routine from the software package S-Plus, MathSoft, Inc., Cambridge, Mass.).

In some specific embodiments, genes are grouped according to the degree of co-variation of their transcription, presumably co-regulation, as described in U.S. Pat. No. 6,203,987. Groups of genes that have co-varying transcripts are termed “genesets.” Cluster analysis or other statistical classification methods may be used to analyze the co-variation of transcription of genes in response to a variety of perturbations, e.g., caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

In addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In certain embodiments, the data obtained from such experiments reflects the relative expression of each gene represented in the microarray. Expression levels in different samples and conditions may now be compared using a variety of statistical methods.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

5.c. Gene Cloning

Cellular compositions of the present invention and progenitor cells and progeny cells derived therefrom, may be engineered into reagent cells, or host cells, suitable for gene cloning methods. Furthermore, methods of the present invention may be incorporated into methods for producing such reagent or host cells and into gene cloning methods. Methods for producing such recombinant reagent cells are well-known in the art. For example, ligating a polynucleotide coding sequence into a gene construct, such as an expression vector, and transforming or transfecting into eukaryotic hosts are standard procedures used in producing other well-known proteins, including sequences encoding exogenous receptor and peptide libraries. Similar procedures, or modifications thereof, can be employed to prepare recombinant reagent cells of the present invention by tissue-culture technology in accord with the subject invention. Cells may be transformed or transfected before, during, or after production of embryoid bodies by the methods of the present invention.

In general, it will be desirable that the vector be capable of replication in the host cell. It may be a DNA which is integrated into the host genome, and thereafter is replicated as a part of the chromosomal DNA, or it may be DNA which replicates autonomously, as in the case of a plasmid. In the latter case, the vector will include an origin of replication which is functional in the host. In the case of an integrating vector, the vector may include sequences which facilitate integration, e.g., sequences homologous to host sequences, or encoding integrases.

Representative examples of vectors which may be used include viral vectors, phage, plasmids, phagemids, cosmids, phosmids, mammalian artificial chromosomes (MAC), and any other vectors suitable for a specific host cell and capable of stably maintaining and expressing a genomic DNA insert of at least 20 kb, and more preferably greater than 50-75 kb.

Appropriate cloning and expression vectors for use with bacterial, fungal, yeast, and mammalian cellular hosts are known in the art, and are described in, for example, Powels et al. (Cloning Vectors: A Laboratory Manual, Elsevier, New York, 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

Certain mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in eukaryotic cells. Alternatively, derivatives of viruses such as the bovine papillomavirus (BPV-1), or Epstein-Barr virus (pHEBo, pREP-derived-and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17.

Preferred vectors for the present invention are the so-called artificial chromosomes. One feature of these vectors is their ability to carry large genetic inserts, e.g., greater than 50 kb, with enough mitotic and meiotic stabilities to make their genetic manipulation straightforward. The upper limit on the size of the insert is often great enough that thousands of genes can be included on one vector.

In certain preferred embodiments, the vector is a mammalian artificial chromosome. Exemplary MACs are described in, for example, U.S. Pat. Nos. 5,721,118 and 6,077,697, as well as Csonka et al., J. Cell Sci., 113:3207-16 (2000); Ebersole et al., Hum. Mol. Genet., 9:1623-31 (2000); deJong et al., Cytometry, 35:129-33 (1999); and Schindelhauer, Bioessays, 21(1):76-83 (1999).

Another artificial chromosome which can be adapted for use in the present invention is the baculovirus artificial chromosomes, such as described in detail in U.S. Pat. No. 6,090,584. The '584 patent discloses a baculovirus artificial chromosome which has the lef-8 gene inactivated. The baculovirus artificial chromosome allows the cloning and expression of heterologous genes in insect and mammalian cells.

The transcriptional and translational control sequences in expression vectors to be used in transforming mammalian cells may be provided by viral sources. For example, commonly used promoters and enhancers are derived from Polyoma, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al., Nature, 273:111 (1978)) Smaller or larger SV40 fragments may also be used, provided the approximately 250 bp sequence extending from the Hind III site toward the Bgl I site located in the viral origin of replication is included. Exemplary vectors can be constructed as disclosed by Okayama and Berg, Mol. Cell Biol., 3:280 (1983). A useful system for stable high level expression of mammalian receptor cDNAs in C127 murine mammary epithelial cells can be constructed substantially as described by Cosman et al., Mol. Immunol., 23:935 (1986). Other expression vectors for use in mammalian host cells are derived from retroviruses.

In other embodiments, the use of viral transfection can provide stably integrated copies of the expression construct. In particular, the use of retroviral, adenoviral or adeno-associated viral vectors is contemplated as a means for providing a stably transfected cell line which expresses an exogenous receptor, and/or a polypeptide library.

The vector should, as pointed out above, include at least one origin of replication for the host cell into which the vector is to be transfected. If also necessary, the vector can include one or more copy-control sequence for controlling the number of copies of the vector in any one cell.

The vector is transfected into and propagated in the appropriate host. Methods for transfecting the host cells with the vector can be readily adapted from procedures that are known in the art. For example, the vector can be introduced into the host cell by such techniques as electroporation, precipitation with DEAE-Dextran or calcium phosphate, or lipofection.

Coding sequences for a polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunological carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, European Patent Publication No. 0259149; and Evans et al., Nature, 339:385 (1989); Huang et al., J. Virol. 62:3855 (1988); and Schlienger et al., J. Virol., 66:2 (1992)).

Fusion proteins may facilitate the expression and/or purification of proteins. For example, a polypeptide of the present invention may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y., John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., J. Chromatography, 411:177 (1987); and Janknecht et al., PNAS, 88:8972-76 (1991)).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons, 1992).

5.d. Model Systems

Cellular compositions of the present invention, as well as progenitor cells and progeny cells derived therefrom, may be engineered into cell lines or transgenic animals suitable for use in methods of modeling various disorders and conditions. Models of the present invention allow studies at the cellular level as well as at the in vivo level. The methods provided by the present invention may be incorporated into methods for producing such model cell lines or animals. For example, ES cells containing homozygous or heterozygous mutations of specific genes may be produced by methods incorporating the methods of the present invention.

Such cell lines or animals may provide models for various genetic disorders, or models with which to study the importance or role of a gene. The cells may also be deficient in the expression of multiple genes, and thus provide the opportunity to study the interactions of proteins with other proteins or the effects these proteins have on gene expression. Such cells may be useful in studying any aspect of gene expression or tissue specific expression of various genes.

Model cell lines and animals of the present invention may be developed for in vivo analysis of the compounds identified by the screening methods of the present invention. The most promising compounds selected from in vitro analysis as described above may be tested in such models for prophylactic and therapeutic efficacy. For example, cellular compositions of the invention may be genetically altered to be predisposed to a developmental disorder. The composition may be analyzed for its ability to prevent the disorder in the cellular compositions as they develop, as well as for any potential side effects. The absence of one or more genes in a cell line or animal allows one skilled in the art to screen for genes and agents which can restore the altered cells or animals to a wild-type phenotype, as well as to screen for agents which act as agonists or antagonists of one or more genes. Thus, it will be appreciated that there are many uses to which the mice and cell lines of the present invention may be put.

As used herein, a transgene is a recombinant nucleic acid molecule that has been introduced into the genome of the animal at the embryonic stage of the animal's development. A transgene that has been incorporated into the genome of an animal is a recombinant nucleic acid molecule that has stably integrated into the DNA of all of the germ cells and somatic cells in an animal. A transgene typically also includes regulatory sequences, such as expression control sequences (e.g., promoters), which control the expression of the transgene in the cells of the animal. Such sequences are described in detail below. A target gene promoter can be isolated, e.g., by screening of a genomic library with an appropriate cDNA fragment and characterized according to methods known in the art. Yet other non-human animals within the scope of the invention include those in which the expression of the endogenous target gene has been mutated or “knocked out”. A “knock out” animal is one carrying a homozygous or heterozygous deletion of a particular gene or genes. These animals could be useful to determine whether the absence of the target will result in a specific phenotype, in particular whether these animals have or are likely to develop a specific disease. Furthermore, these animals are useful as negative controls in screens for drugs which alleviate or attenuate the disease condition, or for gene expression studies in order to determine the genes involved in a disease. The target knock out animals can be crossed with transgenic animals expressing, e.g., a mutated form or allelic variant of the target gene, thereby resulting in an animal which expresses only the mutated protein and not the wild-type target gene product. These animals are also useful for determining the effect of a specific amino acid difference, or allelic variation, in a target gene.

According to the present invention, in general, a transgenic mouse is a mouse which includes a recombinant nucleic acid molecule (i.e., transgene) that has been introduced into the genome of the mouse at the embryonic stage of the mouse's development. As such, the transgene will be present (i.e., incorporated into) in essentially all of the germ cells and somatic cells of the mouse. Methods for the introduction of a transgene into a mouse embryo are known in the art and are described in detail in Hogan et al., Manipulating the Mouse Embryo: A Laboratory Manual, Cold Spring Harbor Press, Cold Spring Harbor, N.Y., 1986. Many U.S. patents also describe production of a transgenic animal (See for example, Leder et al., U.S. Pat. No. 4,736,866; Cordell, U.S. Pat. No. 5,387,742; Lonberg et al., U.S. Pat. No. 5,545,806; Capecchi et al., U.S. Pat. No. 5,487,992; Hammer et al., U.S. Pat. No. 5,489,742; Bleck et al., U.S. Pat. No. 5,530,177; Wheeler, U.S. Pat. No. 5,523,226; Robinson et al., U.S. Pat. No. 5,489,743; Krimpenfort et al., U.S. Pat. No. 5,434,340; and Terhorst et al., U.S. Pat. No. 5,530,179). For example, a recombinant nucleic acid molecule (i.e., transgene) can be injected into the male pronucleus of a fertilized mouse egg to cause one or more copies of the recombinant nucleic acid molecule to be retained in the cells of the developing mouse. A mouse retaining the transgene, also called a “founder” mouse, usually transmits the transgene through the germ line to the next generation of mice, establishing transgenic lines. According to the present invention, a transgenic mouse also includes all progeny of a transgenic mouse that inherit the transgene.

As used herein, a transgene-negative littermate is a mouse which is born into the same litter as a transgenic mouse described herein (i.e., a littermate), but did not inherit the transgene (i.e., is transgene-negative, or does not have the transgene incorporated into its genome). Such a mouse is essentially a normal, or wild-type, mouse and is useful as an age-matched control for the methods described herein.

According to the present invention, any non-human animal may be used as a starting organism for the derivation of a transgenic animal of the present invention. Preferably, a transgenic model of the present invention is a mammal including, but not limited to, rabbits, primates and rodents. Most preferably, a transgenic model of the present invention is a rodent, for example, a mouse.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a specific gene of interest in ES cells, these changes can be introduced into the germlines of animals to generate chimeras. The gene targeting procedure is accomplished by introducing into tissue culture cells a DNA targeting construct that includes a segment homologous to a target locus, and which also includes an intended sequence modification to the genomic sequence (e.g., insertion, deletion, point mutation). The treated cells are then screened for accurate targeting to identify and isolate those which have been properly targeted.

Target gene function may be disrupted through the use of a targeting transgene construct designed to undergo homologous recombination with one or more target genomic sequences. The targeting construct can be arranged so that, upon recombination with an element of a target gene, a positive selection marker is inserted into (or replaces) coding sequences of the gene. The inserted sequence functionally disrupts the target gene, while also providing a positive selection trait. Exemplary targeting constructs are described in more detail below.

Generally, the ES cells used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al., J. Embryol Exp. Morphol., 87:27-45 (1985)). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (loffe et al., PNAS, 92:7357-61 (1995)). The cells are cultured and prepared for knockout construct insertion using methods well known to the skilled artisan, such as those set forth by Robertson in: Teratocarcinomas and Embryonic Stem Cells: A Practical Approach, E. J. Robertson, ed., IRL Press, Washington, D.C. (1987); by Bradley et al., Current Topics in Devel. Biol., 20:357-71 (1986)); and by Hogan et al., Manipulating the Mouse Embryo: A Laboratory Manual, Cold Spring Harbor Laboratory Press, Cold Spring Harbor, N.Y. (1986).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits some coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, a variation of the “knock-in” type of construct can be used to insert a human sequence for expression rather than the host species sequence, or to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., β-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art, i.e., one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker. Nonhomologous recombination between the resulting knock out construct and the genome will usually result in the stable integration of one or both of these negative selectable marker genes and hence cells which have undergone nonhomologous recombination can be selected against by growth in the appropriate selective media (e.g., media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques, PCR, or the like, all of which are well known to those skilled in the art.

Each knockout construct to be inserted into the cell must first be in the linear form. Therefore, if the knockout construct has been inserted into a vector (described infra), linearization is accomplished by digesting the DNA with a suitable restriction endonuclease(s) selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al., infra.

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northem blots can be used to probe the mRNA for the presence or absence of transcripts encoding either the gene knocked out, the marker gene, or both. In addition, Western blots can be used to assess the level of expression of the target gene knocked out in various tissues of the offspring by probing the Western blot with an antibody against the particular target protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo: A Laboratory Manual, (Cold Spring Harbor Laboratory Press, Cold Spring Harbor, N.Y., 1986). Recombinase dependent knockouts can also be generated, e.g., by homologous recombination to insert target sequences, such that tissue specific and/or temporal control of inactivation of a target-gene can be controlled by recombinase sequences.

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A target transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a target protein can be essential for many forms of lineage analysis and can additionally provide a means to assess the effects of, for example, lack of target expression which might grossly alter development in small patches of tissue within an otherwise normal embryo. Towards this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes that can be regulated via site-specific genetic manipulation in vivo, are known to those skilled in the art. It may be appreciated by one skilled in the art that use of recombinant DNA technologies can improve expression of transgenes disclosed herein by manipulating, for example, the number of copies of the nucleic acid molecules integrated into a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, substitutions or modifications of expression control signals (e.g., promoters, operators, enhancers), substitutions or modifications of translational control signals (e.g., ribosome binding sites, Shine-Dalgarno sequences) and deletion of sequences that destabilize transcripts.

For instance, in certain embodiments, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target proteins. For example, excision of a target sequence which interferes with the expression of a recombinant target gene, such as one which encodes an antagonistic homolog or an antisense transcript, can be designed to activate expression of that gene. This interference with expression of the protein can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment that is not intended to be limiting, either the cre/loxP recombinase system of bacteriophage P1 (Lakso et al., PNAS, 89:6232-36 (1992); Orban et al., PNAS, 89:6861-65 (1992)) or the FLP recombinase system of Saccharomyces cerevisiae (O'Gorman et al., Science, 251:1351-1355 (1991); PCT Patent Application Publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., J. Biol. Chem., 259:1509-14 (1984)); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant target protein can be regulated via control of recombinase expression.

Use of the cre/loxP recombinase system to regulate expression of a recombinant target protein requires the construction of a transgenic animal containing transgenes encoding both the Cre recombinase and the subject protein. Animals containing both the Cre recombinase and a recombinant target gene can be provided through the construction of “double” transgenic animals. A convenient method for providing such animals is to mate two transgenic animals each containing a transgene, e.g., a target gene and recombinase gene.

One advantage derived from initially constructing transgenic animals containing a target transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic target transgene is silent will allow the study of progeny from that founder in which disruption of target mediated induction in a particular tissue or at certain developmental stages would result in, for example, a lethal phenotype.

Similar conditional transgenes can be provided using prokaryotic promoter sequences which require prokaryotic proteins to be simultaneously expressed in order to facilitate expression of the target transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g., a recombinase or a prokaryotic protein, is delivered to the tissue and caused to be expressed, such as in a cell-type specific manner. By this method, a Target A transgene could remain silent into adulthood until “turned on” by the introduction of the trans-activator.

A third type of target cell for transgene introduction is the embryonic stem cell (ES). ES cells are obtained from pre-implantation embryos cultured in vitro and fused with embryos (Evans et al., Nature, 292:154-56 (1981); Bradley et al., Nature, 309:255-58 (1984); Gossler et al., PNAS, 83:9065-69 (1986); and Robertson et al., Nature, 322:445-48 (1986)). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, Science, 240:1468-74 (1988).

The tissues of the animal which is the object of this invention can be utilized both as sources of cells for in vitro culture by means of standard culturing techniques, as well as “samples” within the meaning given it herein. Culturing techniques make it possible to obtain primary cultures which can be utilized directly as nontransformed lines for the screening of substances with activity, or can be transformed in order to obtain lines whose cells continue to proliferate, e.g., in an immortalized cell line. These cultures can be used, for example, in the screening of compounds or for gene expression analysis, according the methods of the present invention. Such primary and immortalized cell cultures comprise the “cell lines” of the present invention.

Alternatively, ES cells may be engineered to overexpress certain exogenous or endogenous genes. Furthermore, ES cell lines may be treated with a particular compound during production, e.g., by the methods of the present invention, or infected with a given pathogen, so that the effect of said compound or pathogen on the ES cells may be evaluated. Such treatment or infection may be performed on a cellular composition of the present invention. In one embodiment, the models produced by the methods and compositions of the invention may be used to evaluate the role of a particular gene in an ES cell or its progeny. For example, the mitogen-activated protein (MAP) kinase p38 is a key component of a stress response pathway, and is a target of antiinflammatory drugs. Its biological function was evaluated in a study using ES cells in which a p38 −/− ES cell line was produced (Allen et al., J. Exp. Med., 191:859-69 (2000)). The p38 −/− cells were exposed to chemical stress inducers, and their gene expression evaluated to determine the role of p38 in the stress response.

5.e. Transplants and Cell Replacement Therapy

It is an object of the present invention to provide improved methods for the isolation and propagation of stem cells from embryoid bodies. Such stem cells can then be used, for example, for direct transplantation or to produce differentiated cells in vitro for transplantation or for cell transplantation therapy. The invention accordingly provides, for example, embryoid bodies that may serve as a source for many other, more differentiated cell types such as pancreatic beta cells. Advantages lie in obviating the need for physical dissociation of tissue in order to obtain differentiated cells for various uses, and the potential for greater reproducibility and control of the process.

In one embodiment, a method of preparing cells suitable for cell replacement therapy comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating the resulting embryoid bodies, inoculating the dissociated embryoid bodies into a medium suitable for accomplishing further differentiation, and culturing said dissociated embryoid bodies to a differentiation state suitable for use in a cell replacement therapy. In other embodiments a method of preparing cells suitable for cell replacement therapy comprises preparing EB of a uniform phenotype, and culturing said EB to a differentiation state suitable for use in a cell replacement therapy.

In another embodiment, a method of producing tissue suitable for tissue transplantation comprises inoculating a culture vessel with a culture of undifferentiated embryonic stem cells, wherein said culture vessel contains a medium suitable for inducing embryoid body formation, incubating said culture vessel while subjecting it to shaking, continuing said incubating until embryoid bodies are produced, dissociating the resulting embryoid bodies, inoculating the dissociated embryoid bodies into a medium suitable for accomplishing further differentiation, and culturing said dissociated embryoid bodies to produce a tissue. In another embodiment, a method of producing tissue suitable for tissue transplantation comprises the steps of preparing EB of a uniform phenotype, dissociating the EB, inoculating the dissociated embryoid bodies into a medium suitable for accomplishing further differentiation, and culturing said dissociated embryoid bodies to produce a tissue.

Accordingly, another aspect of the present invention pertains to the progeny of the subject progenitor cells, e.g., those cells which have been derived from the cells of the initial explant culture. Such progeny can include subsequent generations of progenitor cells, as well as lineage committed cells generated by inducing differentiation of the subject progenitor cells after their isolation from the explant, e.g., induced in vitro.

Another aspect of the invention relates to cellular compositions enriched for embryoid bodies, or the progeny thereof. In certain embodiments, the cells will be provided as part of a pharmaceutical preparation, e.g., a sterile, free of the presence of unwanted virus, bacteria and other (human) pathogens, as well as pyrogen-free preparation. That is, for human administration, the subject cell preparations should meet sterility, pyrogenicity, general safety and purity standards as required by FDA Office of Biologics standards.

In certain embodiments, such cellular compositions can be used for transplantation into animals, preferably mammals, and even more preferably humans. The cells can be autologous, allogeneic or xenogeneic with respect to the transplantation host.

Yet another aspect of the present invention provides cellular compositions which include, as a cellular component, substantially pure preparations of the subject embryoid body, or the progeny thereof. Cellular compositions of the present invention include not only substantially pure populations of the progenitor cells, but can also include cell culture components, e.g., culture media including amino acids, metals, coenzyme factors, as well as small populations of non-progenitor cells, e.g., some of which may arise by subsequent differentiation of isolated progenitor cells of the invention. Furthermore, other non-cellular components include those which render the cellular component suitable for support under particular circumstances, e.g., implantation, e.g., continuous culture.

As common methods of administering the embryoid bodies, progenitor cells, and progeny cells of the present invention to subjects, particularly human subjects, which are described in detail herein, include injection or implantation of the cells into target sites in the subjects, the cells of the invention can be inserted into a delivery device which facilitates introduction by injection or implantation of the cells into the subjects. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The progenitor cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating progenitor cells as described herein in a pharmaceutically acceptable carrier or diluent and, as required, other ingredients enumerated above, followed by filtered sterilization.

Support matrices in which the progenitor cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See e.g., U.S. Pat. No. 4,298,002 and U.S. Pat. No. 5,308,701. These matrices provide support and protection for the fragile progenitor cells in vivo and are, therefore, the preferred form in which the progenitor cells are introduced into the recipient subjects.

The present invention also provides substantially pure progenitor cells which can be used therapeutically for treatment of various disorders associated with insufficient functioning of the pancreas.

To illustrate, the subject progenitor cells can be used in the treatment or prophylaxis of a variety of pancreatic disorders, both exocrine and endocrine. For instance, the progenitor cells can be used to produce populations of differentiated pancreatic cells for repair subsequent to partial pancreatectomy, e.g., excision of a portion of the pancreas. Likewise, such cell populations can be used to regenerate or replace pancreatic tissue loss due to, pancreatolysis or pancreatitis.

More recently, tissue-engineering approaches to treatment have focused on transplanting healthy pancreatic islets, typically encapsulated in a membrane to avoid immune rejection. Three general approaches have been tested in animal models. In the first, a tubular membrane is coiled in a housing that contained islets. The membrane is connected to a polymer graph that in turn connects the device to blood vessels. By manipulation of the membrane permeability, so as to allow free diffusion of glucose and insulin back and forth through the membrane, yet block passage of antibodies and lymphocytes, normoglycemia was maintained in pancreatectomized animals treated with this device (Sullivan et al., Science, 252:718 (1991)).

In a second approach, hollow fibers containing islet cells were immobilized in the polysaccharide alginate. When the device was placed intraperitoneally in diabetic animals, blood glucose levels were lowered and good tissue compatibility was observed (Lacey et al., Science, 254:1782 (1991)).

Finally, cells have been placed in microcapsules composed of alginate or polyacrylates. In some cases, animals treated with these microcapsules maintained normoglycemia for over two years (Lim et al., Science, 210:908 (1980); O'Shea et al., Biochim. Biophys. Acta., 840:133 (1984); Sugamori et al., Trans. Am. Soc. Artif. Intern. Organs, 35:791 (1989); Levesque et al., Endocrinology, 130:644 (1992); and Lim et al., Transplantation, 53:1180 (1992)). However, all of these transplantation strategies require a large, reliable source of donor islets.

The progenitor cells of the invention can be used for treatment of diabetes because they have the ability to differentiate into cells of pancreatic lineage, e.g., β islet cells. The progenitor cells of the invention can be cultured in vitro under conditions which can further induce these cells to differentiate into mature pancreatic cells, or they can undergo differentiation in vivo once introduced into a subject. Many methods for encapsulating cells are known in the art. For example, a source of β islet cells producing insulin is encapsulated in implantable hollow fibers. Such fibers can be pre-spun and subsequently loaded with the 8 islet cells (Aebischer et al., U.S. Pat. No. 4,892,538; Aebischer et al., U.S. Pat. No. 5,106,627; Hoffman et al., Expt. Neurobiol., 110:3944 (1990); Jaeger et al., Prog. Brain Res., 82:4146 (1990); and Aebischer et al., J. Biomech. Eng., 113:178-83 (1991)), or can be co-extruded with a polymer which acts to form a polymeric coat about the β islet cells (Lim, U.S. Pat. No. 4,391,909; Sefton, U.S. Pat. No. 4,353,888; Sugamori et al., Trans. Am. Artif. Intern. Organs, 35:791-99 (1989); Sefton et al., Biotechnol. Bioeng., 29:113543 (1987); and Aebischer et al., Biomaterials, 12:50-55 (1991)).

In certain embodiments, the cellular compositions of the invention may also be used for the treatment of, for example, diseases of the liver, or disorders of insufficient production of blood cells, such as thrombocytopenias, anemias or for transplanting into radiation therapy patients.

Moreover, in addition to providing a source of implantable cells, either in the form of the progenitor cell population of the differentiated progeny thereof, the subject cellular compositions can be used to produce cultures of cells for production and purification of secreted factors. For instance, cultured pancreatic cells can be provided as a source of insulin. Likewise, exocrine cultures can be provided as a source for pancreatin.

### Exemplification

The invention now being generally described, it will be more readily understood by reference to the following examples which are included merely for purposes of illustration of certain aspects and embodiments of the present invention, and are not intended to limit the invention in any way.

### EXAMPLE 1

### Suspension Culture of EBs from DBA252 Murine Embryonic Stem Cells and Subsequent Large-Scale In Vitro Differentiation into Macrophages

Overview

The DBA252 murine embryonic stem cell line was maintained in the undifferentiated state by continuous subculture in media containing murine myeloid leukemia inhibitory factor (mLIF). Two days prior to the beginning of the differentiation process, the cells were passed from SCML, a medium containing mLIF with Knock-Out DMEM as a base, to I/SCML, a medium containing mLIF with Iscove's Modified DMEM as a base. The media change prior to differentiation allowed the ES cells to adapt to the new base medium used throughout the remaining steps of the differentiation, but still maintained the culture in the undifferentiated state.

The differentiation process was as follows. ES cells grown in I/SCML were harvested and seeded into mLIF-free MAC EB medium at day=0. The formation of EB commenced after this seeding, resulting in early differentiation into various cell lineages. Following 6 days of EB formation in MAC EB medium, the EBs were dissociated by trypsinization. The cells from the dissociated EBs were then transferred to MAC I medium containing murine interleukin 3 (IL-3) and macrophage colony stimulating factor (M-CSF). During the first several days of culture in MAC I medium, a number of different cell types were observed under the microscope. As the MAC I culture was subcultured and expanded, these other cell types were gradually reduced in favor of cells in the pre-macrophage lineage. The culture in MAC I medium was always maintained in the confluent state, including the days immediately following the initial inoculation into MAC I after EB dissociation, as well as after subculture in MAC I. As pre-macrophages proliferated in the MAC I culture, they lifted from the monolayer of cells and floated as nonadherent cells in the culture medium. The confluence of the culture prohibited the nonadherent macrophages from adhering and contributing to the further formation of the monolayer. The confluence of the culture additionally facilitated the harvest when spent medium was removed from the culture and replaced with fresh MAC I. Nonadherent cells collected from MAC I were cultured in MAC II and allowed to fully mature to macrophages or cryopreserved for use at a later time. Further proliferation in MAC II was limited by the absence of IL-3 in the medium.

Premature nonadherent cells collected from the MAC I culture or mature macrophages collected from the MAC II culture were analyzed by FACS analysis to confirm the desired macrophage phenotype. Macrophages were typically collected from the MAC I culture on a two to three day cycle from day 14 to day 30. The best yield with correct phenotypic morphology is normally observed between day 17 and 24. Details of this large-scale differentiation method follow. Table 1 contains information about the origin and composition of the reagents and media used in the following protocols.

TABLE 1

| Reag nt/ | | |
|---|---|---|
| Medium | Supplier | Components |
| trypsin/ | Invitrogen | 0.05% trypsin |
| EDTA | | 10 mM EDTA |
| Dulbec- | Invitrogen | |
| co's | | |
| PBS | | |
| SCML | see | For 600 mL: |
| culture | components | 500 mL Knock-out DMEM (Invitrogen) |
| medium | | 90 mL ES Cell Qualified FBS (Invitrogen) |
| | | 6 mL 200 mM L-glutamine stock (Invitrogen) |
| | | 6 mL MEM non-essential amino acids |
| | | (Invitrogen) |
| | | 4 μL beta-mercaptoethanol (Sigma Cell |
| | | Culture) |
| | | 60 μL mLIF (ESGRO, 1E07 unit/mL, |
| | | Chemicon) |
| | | 1.5 mL gentamycin solution (Invitrogen) |
| I/SCML | see | For 600 mL: |
| culture | components | 500 mL Iscove's Modified DMEM (Invitrogen) |
| medium | | 90 mL ES Cell Qualified FBS (Invitrogen) |
| | | 6 mL 200 mM L-glutamine stock (Invitrogen) |
| | | 6 mL MEM non-essential amino acids |
| | | (Invitrogen) |
| | | 4 μL beta-mercaptoethanol (Sigma Cell |
| | | Culture) |
| | | 60 μL mLIF (ESGRO, 1E07 unit/mL, |
| | | Chemicon) |
| | | 1.5 mL gentamycin solution (Invitrogen) |
| Mac EB | see | For 600 mL: |
| culture | components | 500 mL Iscove's Modified DMEM (Invitrogen) |
| medium | | 90 mL ES Cell Qualified FBS (Invitrogen) |
| | | 6 mL 200 mM L-glutamine stock (Invitrogen) |
| | | 45 mL transferrin (4 mg/mL Invitrogen) |
| | | 22.68 μL monothioglycerol (Sigma, to 400 μM) |
| | | 3 mL Ascorbic Acid (10 mg/mL stock, Sigma) |
| | | 30 mL Protein Free Hybridoma Medium |
| | | (Invitrogen) |
| | | 1.5 mL gentamycin solution (Invitrogen) |
| MAC I | see | For 600 mL: |
| culture | components | 500 mL Iscove's Modified DMEM (Invitrogen) |
| medium | | 90 mL ES Cell Qualified FBS (Invitrogen) |
| | | 6 mL 200 mM L-glutamine stock (Invitrogen) |
| | | 22.68 μL monothioglycerol (Sigma, to 400 μM) |
| | | 20 μL M-SCF (0.156 μg/μL stock, R&D |
| | | systems) |
| | | 10 μL IL-3 (62.5 ng/μL stock, R&D systems) |
| | | 1.5 mL gentamycin solution (Invitrogen) |
| MAC II | see | For 600 mL: |
| culture | components | 500 mL Iscove's Modified DMEM (Invitrogen) |
| medium | | 90 mL ES Cell Qualified FBS (Invitrogen) |
| | | 6 mL 200 mM L-glutamine stock (Invitrogen) |
| | | 22.68 μL monothioglycerol (Sigma, to 400 μM) |
| | | 20 μL M-SCF (0.156 μg/μL stock, R&D |
| | | systems) |
| | | 1.5 mL gentamycin solution (Invitrogen) |

Subculture of ES Cells in SCML with mLIF

The DBA252 ES cells were cultured in suspension in SCML medium in a 100 mm Petri dish at 37° C., 6% CO₂ in humidity, with daily feeding and colony morphology monitoring. Healthy ES cell colonies have smooth borders, are tightly packed together so individual cells are not detectable, and their colony has enough depth to give a refractile ring around it. Once a monolayer formed, the medium was removed and the monolayer washed with Dulbecco's PBS (Invitrogen). Pre-warmed 0.05% trypsin/EDTA was added, and the monolayer was incubated at 37° C. for 2-3 minutes. Subsequently, the condition of the culture was evaluated to see if the cells were detached by looking to see if they had rounded up. The culture was incubated for additional 30 second intervals until the cells became detached. The culture was not allowed to remain in the trypsin for more than several additional minutes, as the trypsin causes cell lysis if incubated with cells too long. Once the cells detached or became nonadherent, fresh SCML was added to the culture. The resulting cell suspension was pipetted vigorously with a glass serological pipette to break up remaining clumps. The cells were subsequently transferred to a 50 mL conical tube and centrifuged for 5 minutes at 235×g at room temperature. The medium was aspirated from the cell pellet. The cell pellet was dissociated and mixed in fresh SCML. The cells were inoculated at a density of 1×10⁵ cells/mL into tissue culture Petri dishes or T175 flasks containing SCML medium. The cells were incubated at 37° C., with the medium changed the following day.

Embryoid Body Formation

One passage of the cells as described above before the commencement of EB formation and cell differentiation, the cells were passed into I/SCML and incubated overnight at 37° C., 6% CO₂. The following day the medium was exchanged for fresh I/SCML, and incubated for one more day. At that time, the cells were harvested as described in the previous section. The cell pellet was dissociated and resuspended into MAC EB medium. The suspension was used to inoculate a 3-L Fernbach culture flask containing MAC EB medium at a density of 0.5 to 1.0×10⁵ cells/mL. The inoculation time point was marked as t=0 of the differentiation. The Fernbach flask culture was incubated at 37° C., 6% CO₂ on a rotary shaker at 50 RPM. After three days, the culture was removed from the shaker and the EBs allowed to settle to the bottom corner of the Fernbach flask. The spent medium was aspirated until only 50 mL of medium remained, and a fresh 1L of MAC EB medium was added. The culture was again incubated at 37° C., 6% CO₂ on a rotary shaker at 50 RPM. After two more days (five days from initial inoculation of the culture), the EBs again were allowed to settle and the medium was exchanged.

Dissociation of EBs

Six days post-inoculation of the Fernbach flask culture, the EBs were allowed to settle to the bottom corner of the flask. The medium was aspirated off until 50 mL of medium remained, then the EB/medium suspension was transferred into two 250 mL conical tubes in equal aliquots. The EBs were allowed to settle and the remaining medium was aspirated off. 200 mL of Dulbecco's PBS were added and mixed with the EBs. The EBs were allowed to settle, and the PBS was aspirated off. The PBS wash step was repeated, then the EBs were resuspended in 200 mL of prewarmed 0.05% trypsin EDTA. The suspensions were transferred to a 250 mL Sigmacoat-siliconized spinner flask. The coated flask had been washed with Sigmaclean® cell culture glassware detergent, rinsed with hydrogen peroxide, rinsed with pyrogen-free 18 megaohm water and autoclaved for 30 minutes at 121° C. prior to use. The culture was incubated in the spinner flask at 37° C., 6% CO₂ on a spinner plate at 200-300 RPM for 7-10 minutes until most of the EBs were dissociated. The dissociated suspension was transferred into four 250 mL conical tubes. 150 mL of MAC I medium were added to each tube to inactivate the trypsin. The cell suspensions were centrifuged at 235×g for 5 minutes. The medium was aspirated from the cell pellet, which was subsequently disrupted and resuspended in fresh MAC I.

Cell Factory Inoculation

The cell suspension resulting from the procedure in the above section was transferred to a 2L aspirator bottle fitted with tubing and connectors, resulting in a total volume of approximately 800 mL. The aspirator bottle was assembled per the Nunclon®. Cell Factory manufacturer's protocol and had been washed with Sigmaclean® cell culture glassware detergent, rinsed with hydrogen peroxide, rinsed with pyrogen-free 18 megaohm water and autoclaved for 30 minutes at 121° C. prior to use. MAC I medium was added to a final volume of 1.3L and the suspension mixed. The cell number per mL and cell viability were determined to be 2.5 to 5×10⁵ viable cells/mL and >70% viability. 40 mL of the suspension were removed and transferred to a T175 flask as a control. A presterilized Acrodisc vent filter was attached to one of the sterile connectors of the Cell Factory per the manufacturer's protocol. The aspirator bottle was attached to the Cell Factory and the cell suspension was fed into the cultureware per the manufacturer's protocol. The aspiratory tubing connector was removed and a second presterilized Acrodisc vent filter was attached in its place. The Cell Factory and control T175 were coinucubated, marking day 6 of the differentiation. The next day, the medium over the monolayer in the Cell Factory was gently swirled and the medium drained per the Cell Factory manufacturer's protocol. Fresh MAC I medium was added, and the Cell Factory returned to the incubator.

Cell Factory Subculture

On day 10 of the differentiation, the Cell Factory was swirled and drained. The spent medium contained nonadherent premature macrophages that were harvested. 500 mL of Dulbecco's PBS was added to a second sterile aspirator bottle and drained into the Cell Factory to rinse the monolayer. The PBS rinse was drained back into the bottle. 100 mL of prewarmed 0.05% trypsin EDTA was added to a third sterile aspirator bottle and drained into the Cell Factory, which was swirled to cover the monolayer in each chamber evenly. The Cell Factory was returned to the incubator for 2-3 minutes with the aspirator tube still attached. After this, the Cell Factory was removed from the incubator and rapped sharply to dislodge the cells. 500 mL fresh MAC I was added via the attached aspirator bottle and drained into the unit to neutralize the trypsin solution. The Cell Factory was swirled, and the suspension drained back into the aspirator bottle. The cell suspension was transferred from the bottle into four 250-mL conical tubes. The tubes were centrifuged at 235×g for 5 minutes. The medium was decanted and the tubes tapped to dislodge the cell pellets, which were each resuspended in 50 mL MAC I. The suspension from two of the four tubes was transferred back into the aspirator bottle already attached to the Cell Factory. The remaining cell suspension was transferred into a new sterile aspiratory bottle and inoculated into a second Cell Factory. Additional MAC I medium was added to each bottle to a total volume of 1.3 L. 40 mL of culture was removed from each bottle and set up in a T175 flask as a control. The cell suspensions were drained into the Cell Factories, and incubated overnight along with the control flasks. The next day, the medium was swirled over the surface of the monolayers and the cell debris removed by draining the spent medium. 1.3 L of fresh MAC I medium was added to each Cell Factory.

Cell Factory Premature Macrophage Harvest

Nonadherent premature macrophages were harvested using the day 14 subculture, then again every two to three days to about day 30 of the differentiation. The conditioned medium containing the premature macrophages was drained into a sterile aspirator bottle, and then aliquotted into 250 mL conical tubes. The monolayer remaining in the Cell Factory was rinsed with 200 mL of PBS to remove more premature macrophages, and this rinse pooled with the premature macrophages already in the 250 mL conical tubes. 1.3 L of fresh MAC I medium was drained into the Cell Factory, which was returned to the incubator. The suspensions in the tubes were centrifuged at 235×G for 5 minutes. The supernatant was decanted, and the cell pellet disrupted in MAC II medium.

### EXAMPLE 2

### Characterization of EB Prepared by Suspension Culture

EB formation was accomplished as described in Example 1. Cells comprising the EBs were characterized by cell size and cell viability from measuring cells released from dissociated EBs, as well as by expression of the hematopoietic cell surface markers CD34 and FLK-1 using the method of Dang, et al., Biotech. Bioeng., 78:442-53 (2002). Measurable characteristics were compared to EB formed in 100 mm microbiological Petri plates, which is the standard method of EB formation known in the art. The results of physical characterization are presented in Table 2, while the results of cell surface marker expression are presented in Table 3. Table 2 indicates that the average cell diameters of cells comprising EB formed by the suspension method, which range from 13.73 to 14.32 microns, are greater than those cells comprising EB formed by the standard Petri dish method, which range from 10.67 to 10.99 microns. Furthermore, the viability of the cells comprising EB formed by the suspension method are on average greater than the viability of the cells comprising EB formed by the standard Petri dish method, a range of 94-98% viability vs. 90-93% viability. Table 3 indicates in general that higher expression of the markers CD34 and FLK-1 was observed in cells comprising EB formed by the suspension method than in cells comprising EB formed by the standard Petri dish method.

TABLE 2

| | | | Avg. cell | |
|---|---|---|---|---|
| | | Starting | diameter | % viable |
| Method | RPM | Density | (microns) | cells in EB |
| suspension | 100 | 0.4 × 10⁵/mL | 14.32 | 96 |
| suspension | 100 | 1.0 × 10⁵/mL | 13.73 | 94 |
| suspension | 150 | 0.4 × 10⁵/mL | 14.47 | 95 |
| suspension | 150 | 1.0 × 10⁵/mL | 13.93 | 97 |
| suspension | 200 | 0.4 × 10⁵/mL | 14.08 | 94 |
| suspension | 200 | 1.0 × 10⁵/mL | 14.04 | 98 |
| Petri dish | N/A | 1.5 × 10⁵/mL | 10.89 | 93 |
| Petri dish | N/A | 1.5 × 10⁵/mL | 10.99 | 92 |
| Petri dish | N/A | 1.5 × 10⁵/mL | 10.67 | 90 |

TABLE 3

| | | Starting | % expressing | % expressing |
|---|---|---|---|---|
| Method | RPM | Density | CD34 | FLK-1 |
| suspension | 100 | 0.4 × 10⁵/mL | 11.3 | 30.11 |
| suspension | 100 | 1.0 × 10⁵/mL | 4.3 | 21.68 |
| suspension | 150 | 0.4 × 10⁵/mL | 11.76 | 18.59 |
| suspension | 150 | 1.0 × 10⁵/mL | 10.48 | 24.73 |
| suspension | 200 | 0.4 × 10⁵/mL | 13.71 | 24.35 |
| suspension | 200 | 1.0 × 10⁵/mL | 9.19 | 19.68 |
| Petri dish | N/A | 1.5 × 10⁵/mL | 3.8 | 19.54 |

### Equivalents

The present invention provides, among other things, methods and compositions for effecting large-scale in vitro differentiation of mammalian ES cells. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

We claim:

1. A method for the formation of embryoid bodies, comprising (a) inoculating a culture vessel with a culture of undifferentiated embryonic stem cells, wherein said culture vessel contains a medium suitable for inducing embryoid body formation; and (b) incubating said culture vessel while subjecting it to shaking.

2. The method of claim 1, wherein said shaking comprises rotary shaking at a rate from about 60 to about 200 RPM.

3. The method of claim 1, wherein said undifferentiated embryonic stem cells are of mammalian origin.

4. The method of claim 3, wherein said undifferentiated embryonic stem cells are of murine origin.

5. The method of claim 3, wherein said undifferentiated embryonic stem cells are of human origin.

6. A method for differentiating embryonic stem cells, comprising: (a) inoculating a culture vessel with a culture of undifferentiated embryonic stem cells, wherein said culture vessel contains a medium suitable for inducing embryoid body formation; (b) incubating said culture vessel while subjecting it to shaking; (c) continuing said incubating until embryoid bodies are produced; (d) dissociating the resulting embryoid bodies; (e) inoculating the dissociated embryoid bodies into a medium suitable for accomplishing further differentiation; and (f) culturing said dissociated embryoid bodies to a later differentiation state.

7. The method of claim 6, wherein said shaking comprises rotary shaking at a rate from about 60 to about 200 RPM.

8. The method of claim 6, wherein said dissociating step comprises trypsinization of the embryoid bodies.

9. The method of claim 6, wherein said medium suitable for accomplishing further differentiation is comprised of at least one molecule that accomplishes final differentiation of said embryoid bodies.

10. The method of claim 9, wherein said culturing step results in the formation of a differentiated embryonic stem cell.

11. The method of claim 10 wherein said differentiated stem cell is selected from the group consisting of: macrophages, hepatocytes, pancreatic cells, neural cells, cardiac cells, smooth muscle cells, pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, tonsil tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, fetal tissue and umbilical tissue.

12. The method of claim 6, wherein said method further comprises: (g) contacting a differentiated cell produced by said culturing step with a therapeutic agent.

13. The method of claim 6, wherein said method further comprises: (g) using a cell produced by said culturing step in a cell replacement therapy.

14. The method of claim 6, wherein said method further comprises: (g) using a cell produced by said culturing step to produce a tissue.

15. The method of claim 6, wherein said method further comprises: (g) determining the gene expression of the cells or embryoid bodies in the culture in at least one of (a) through (e).

16. The method of claim 6, wherein said method comprises: (g) determining the gene expression in a differentiated cell produced by said culturing step and in an embryoid body produced by said incubating step.

17. A cellular composition of embryoid bodies, wherein at least 80% of cells comprising said embryoid bodies have a diameter from about 13 to about 15 microns.

18. The composition of claim 12 wherein at least 95% of cells comprising said embryoid bodies are viable.

19. The composition of claim 12 wherein at least 10% of cells comprising said embryoid bodies express CD34.

20. A cellular composition of embryoid bodies, wherein at least 20% of cells comprising said embryoid bodies express FLK-1.
